(19) United States
(12) Patent Application Publication
(10) Pub. No.: US 2002/0173036 A1
(43) Pub. Date: Nov. 21, 2002

(54) Cell line and method of making and using same

(76) Inventor: Charleata A. Carter, Little Rock, AR (US)

Correspondence Address: JOE D. CALHOUN, 319 PRESIDENT CLINTON AVENUE, SUITE 205, LITTLE ROCK, AR, 72201, US

(21) Appl. No.: 09/819,193

(22) Filed: Mar. 28, 2001

(51) Int. Cl. G01N 33/574; C12N 5/08

(52) U.S. Cl. 435/366; 435/7.23

(57) ABSTRACT

A cell line isolated from a patient with metastatic adenocarcinoma consistent with primary endometrial cancer. These cells (“CAC-1 cells”) originated from glandular epithelial cells forming a primary tumor of the endometrium of the uterus. In general, these cells are poorly differentiated and highly invasive. Besides manifestations of abnormal karyotype, another distinguishing characteristic may be that typical CAC-1 cells are hyperdiploid, having 48 chromosomes rather than the standard 46 chromosomes; they are triploid at chromosomes 3, 7 and 17, but only haploid at chromosome 14.

### CROSS REFERENCES TO RELATED APPLICATIONS

Not applicable.

### FEDERALLY SPONSORED RESEARCH OR DEVELOPMENT

Not applicable.

### MICROFICHE APPENDIX

Not applicable.

### BACKGROUND OF THE INVENTION

(1) Field of the Invention

The invention described herein generally relates to a cell line (hereinafter “CAC-1 cells”), and more particularly to a cell line isolated from a human endometrial adenocarcinoma; the invention described herein also generally relates to methods of culturing said cells in vitro, and to methods of using said cells.

(2) Description of the Related Art Including Information Disclosed Under 37 C.F.R. 1.97 and 1.98

It is generally understood that cancer cells have essentially the same components as normal counterparts except that cancer cells lack the same regulatory balances maintained by their normal cells. It is these regulatory failures that result in cancer manifested by an aberrant genetic phenotype. At lease three cellular functions tend to be improperly regulated in a neoplasm such as, for example, an adenocarcinoma. Cellular proliferation is unrestrained, accounting for the drastic increase in the number of cells. Cell differentiation can be prevented or stunted, thereby causing individual cells to have distorted shapes or characteristics different from the norm. Moreover, chromosomal and genetic organization may be destabilized.

The genes expressed by a particular cell only comprise approximately 20 to 30 percent of the genes actually present within the cell. Differential gene expression is one aspect of cell differentiation. All cells of a particular type or tissue typically express the same genes, and thereby manifest the same (or similar) appearance and functions; they manifest the same phenotype. Cell differentiation encompasses the ability of each independent individual cell to properly express the genes for a cell of that particular type, rather than manifesting the phenotype for cells of a different normal type (or cells having abnormal phenotypic expression).

A neoplasm is essentially a tissue mass formed from uncontrolled proliferation of cells, usually due to loss of normal cell growth control; these may be either benign or malignant (often invasive). The degree of tumor malignancy is also related to the degree of cell differentiation; undifferentiated or abnormally immature cells, often called anaplastic, are more likely indicative of malignancy. In some abnormal cells such as uterine cancer cells, especially endometrial adenocarcinoma cells, increased cell proliferation and decreased cell differentiation result in the formation of a tumor and/or lesion. An adenocarcinoma is, by definition, a malignant glandular structure. If it is the initial mass of proliferating cells, it is a primary tumor; otherwise, it is a secondary tumor. A lesion is generally a region of abnormal change in the tissue.

Pathologists typically grade tumors on a scale of Grade I to Grade III. Grade I is essentially an almost completely differentiated cell. Grade III represents the least differentiated, fastest dividing cells, presumed to be indicative of tumors having the worst prognosis. Grade II cells are moderately differentiated.

Histologically, most endometrial carcinomas are characterized by more or less well-defined gland patterns lined by malignant cuboidal or columnar epithelial cells. They may be well differentiated (Grade I), having a prominent and easily recognizable glandular pattern. They may also be moderately differentiated (Grade II), where well-formed glands are mixed with solid sheets of malignant cells. Endometrial carcinomas may also be poorly differentiated (Grade III), characterized by solid sheets of cells with barely recognizable glands and a great degree of nuclear atypia and mitotic activity.

Gene expression, cytoskeletal integrity, cell adhesion and differentiation are controlled by the complex interplay of protein kinases and phosphatases in specific signaling pathways. Protein kinases typically contain a 250 amino acid catalytic domain that is commonly under the control of a separate regulatory domain. There are 5 kinase categories:

(1) cyclic nucleotide-regulated and phospholipid-regulated kinases and ribosomal S6 kinases;

(2) the calcium and calmodulin kinases;

(3) the cyclin-dependent kinases;

(4) the protein tyrosine kinases; and

(5) “other” kinases falling outside the 4 major groups.

Members of groups share substrate preferences. For example, both the cyclic nucleotide-regulated and phospholipid-regulated kinases and the calcium/calmodulin kinases phosphorylate serine/threonine residues near arginine and/or lysine. The cyclic nucleotide-regulated and phospholipid-regulated kinases include cyclin nucleotide-dependent protein kinases (protein kinase A and protein kinase G) and lipid-dependent protein kinase C families.

Protein kinase C (“PKC”) is a family of serine/threonine protein kinases consisting of several subfamilies (such as α, β I, β II, γ; novel: δ, ε, θ, η, μ; atypical: λ, ζ, τ) that lie on signal transduction pathways and regulate growth and differentiation in eukaryotic cells [1, 2; these reference numerals, and all other bracketed reference numerals in the Background of the Invention, correspond to the end note numerals appearing in the inventor's paper entitled Protein Kinase C as a Drug Target: Implications for Drug or Diet Prevention and Treatment of Cancer published in Current Drug Targets, 1: 163-183 (2000)]. PKC controls various normal physiological processes and is also involved in tumor promotion and multistage carcinogenesis [3-5].

Because of the recent advances in antisense technology against individual PKC isoforms, studies using new analogs of PKC inhibitors (and new information on naturally occurring PKC inhibitors), some researches now focus on how alterations in PKC may treat or prevent cancer.

PKC isoforms are classified on the basis of their requirement for Ca²⁺, phosphatidylserine and diacylglycerol (DAG) or phorbol esters for activation (reviewed in Refs. 6-8). Conventional PKCs are regulated by DAG, phosphatidylserine and calcium, whereas novel PKCs are calcium independent, but regulated by DAG and phosphatidylserine. DAG is the endogenous PKC activator that is a product of phosphatidylinositol and results from cellular stimulation. Both PKC groups are also activated by tumor-promoting phorbol esters 12-O-tetradecanoylphorbol-13-acetate (TPA) and certain other phorbol esters [9,10]. DAG regulates the function of PKC by dramatically increasing the affinity of PKC for phosphatidylserine-containing membranes. The resulting high affinity membrane interaction leads to a conformational change that activates the enzyme; the autoinhibitory pseodosubstrate domain of the molecule is removed from the active site, thus allowing substrate binding and catalysis [7]. Additional endogenous activators include cis-unsaturated fatty acids and lysphosphatidylcholine. Atypical PKC isoforms are phosphatidylserine-dependent, but calcium, DAG and TPA independent. PKC isoforms consist of a single polypeptide chain that contains an amino-terminal regulatory region (of 20-70 kDa) and a carboxy-terminal kinase domain (of approximately 45 kDa) (Reviewed in 8 and 11). For a review of the structure and regulation of PKC, see Newton, 1997 [7].

Phosphorylation is arapid and reversible means of regulating protein activity. PKC mediates phosphorylation of numerous protein substrates [12-14], and phosphorylation has been shown to play an essential role in regulating PKCs [15,16]. When PKC is activated, it is recruited to membranes upon the production of diacylglycerol and calcium (for conventional isoforms) [1]. PKC autophosphorylates itself in vitro [17]. PKC is multipli-phosphorylated in vivo [15,16]. PKC is activated by the lipid second messenger DAG. Most cells express more than one isoform of PKC, suggesting that individual isoforms have unique rather than overlapping functions. The presence of a number of different PKC isoforms with minor differences in their biochemical characteristics may provide the cell with a way to control a wide spectrum of physiological responses. Varied expression in different cell types, or during alterations in tissue differentiation, might explain how signal transduction via PKC can stimulate various cellular responses.

Evidence suggests that the members of the PKC family are activated in specific intracellular compartments in various ways, depending on different membrane lipid metabolites, and that PKC isoforms play distinct roles in the control of major cellular functions. Multiple lipid mediators have the potential to prolong PKC activation for sustained cellular responses (see Nishizuka, 1995 for review) [18]. Coordinated regulation of PKC activation is crucial for normal cell functions. Twelve different PKC isoforms have been identified. This demonstrates that there are most likely complex pathways by which each PKC isoform exerts its specific function and that our knowledge remains near the beginning of understanding the complexity of the effects of each isoform. Although some variations in PKC function(s) occur between cell and tissue types, some basic information is available on most isoforms.

PKC-α is the most widely expressed PKC isoform. PKC-α and -δ mediate differentiation of mouse myeloid cells into mature macrophages [19]. PKC-α expression regulates the anti-proliferative action of retinoids on human breast carcinoma cells. When retinoic acid induced the arrest of proliferation in human breast carcinoma cells, PKC-α expression increased, concomitant with decreased PKC-ζ expression [20]. Increased PKC activity resulted in enhanced cell growth in myoblasts; treatment with TPA inhibited DNA synthesis and down-regulated PKC-α suggesting that this isoform has a role in maintaining proliferation [21]. In another study, inhibition of PKC-α increased apoptosis, indicating that PKC-α was associated with cell survival and suppressed apoptosis in glioma cells [22]. PKC-α localized exclusively to the cytoplasm when human endometrial adenocarcinoma cells differentiated in response to pharmacological doses of retinoic acid, indicating inactivation of PKC-α during differentiation [23].

Studies indicate that PKC-β II is selectively involved in colonic epithelial cell proliferation and colon carcinogenesis. Overexpression of PKC-β II induces colonic hyperproliferation and increased sensitivity to colon carcinogenesis [24]. PKC-β II is the most responsive of the PKC isoforms expressed in the colonic epithelium to activation by secondary bile acids [25]. Intestinal PKC-β II is resistant to activator-mediated down-regulation that usually occurs in the presence of chronically elevated diacylglycerol, whereas most colonic PKC isoforms (PKC-α, -δ and -ζ) are reduced [26-31]. PKC-β II is also required for proliferation of leukemia cells [32]. PKC-δ is highly expressed in brain and spinal cord and is primarily localized in dendrites and neuron cell bodies.

PKC-δ seems to have a tumor suppressor function [33]. PKC-δ has a role in the execution of the apoptotic program, but PKC-α and ζ are frequently associated with cell survival and suppression of apoptosis [34]. PKC-δ also regulates sodium, potassium, chloride co-transport [35] and inhibits the Stat signaling pathway [36]. PKC-ε overexpression results in tumorigenesis, leading to its classification as an oncogene [37]. PKC-η plays an important role in epithelial differentiation. Activation of PKC-η also inhibits cell growth. PKC-η is relatively highly expressed in skin cells and is thought to mediate transcriptional activation of the human transglutaminase 1 gene, which is expressed during terminal differentiation of keratinized squamous epithelium [38]. PKC-η is upregulated during rat mammary gland differentiation [39]. Overexpression of PKC-η inhibited the growth of keratinocytes of human and mice [40]. Activation of PKC-η alters the cytoskeleton and cell morphology in mouse thyoma cells in a manner similar to that of phorbol ester treatment, suggesting a role for PKC-η in cytoskeletal organization [41]. PKC-θ selectively stimulates the transcription factor complex AP-1 in T lymphocytes and is selectively activated during T-cell activation [42,43]. PKC-θ is implicated in cell cycle progression in mouse thyoma cells [41]. PKC-μ and its murine homolog (protein kinase D) form a distinct class because the kinase core is most similar to that of calmodulin-dependent kinases (reviewed in [44]). PKC-μ appears to associate with the §-cell antigen receptor complex and is involved in regulating lymphocyte signaling [45].

The atypical PKC members (ζ, τ and λ) have some similar sequence homology. PKC-ζ is involved in a wide range of physiological processes including mitogenesis, protein synthesis, cell survival and transcriptional regulation. PKC-ζ is the physiological target of phosphatidylinositol 3-kinase (PI-3-kinase) and its lipid product phosphatidylinositol-3,4,5-P₃ [46]. In endothelial cells, phosphorylation of NF-kappaB occurs through a pathway involving activation of protein kinase C-ζ and p21 ras [47]. PKC-ζ expression can substantially suppress invasion and metastasis [48]. PKC-τ exhibits 72% homology with PKC-ζ. PKC-τ protects human leukemia cells against drug-induced apoptosis [49]. Similar to the τ isoform, PKC-λ exhibits 72% homology with PKC-ζ. The chemopreventitive effects of dietary fish oil and pectin have been associated with the blockage of azoxymethane-induced alterations in colonic PKC-λ protein. In one study, at the 15-week time point, azoxymethane-injected rats fed corn oil had higher levels of membrane PKC-λ realtive to the fish oil and pectin groups [50]. In another study, insulin activated PKC-ζ and PKC-λ, and stimulated their translocation to membrane fractions in rat adipocytes [51].

The potential of PKC as a target for anticancer drugs has been recognized for several years and has been the subject of several reviews [52-55]. When PKC is overexpressed in cells, altered cell function consistent with the transformed phenotype is displayed and likewise elevated PKC levels are often seen in malignant tissues. PKC has been proposed to play a key role in neoplastic transformation because PKC is bound and activated by phorbol esters [56] and PKC phosphorylates the ras oncogene product [57]. In cells transfected with a plasmid encoding PKC, over expression of PKC induced anchorage-independent growth of rat fibroblasts[58] and enhanced the tumorigenicity of murine 3T3 cells [59]. Increased PKC activity, or a change in its subcellular localization, may be related to neoplastic transformation [12, 60]. PKC inhibition in tumors has been associated with tumor growth inhibition during treatment with the staurosporine analogue CGP41251 [61]. Recently, the first report of using a PKC inhibitor to block in vivo signaling pathways in cancer patients showed that the staurosporine analogue CGP41251 acts downstream of PKC because the inhibitor suppressed cytokine release and extracellular signal-regulated kinase 2 expression in cancer patients [62].

PKC activity was significantly higher in endometrial cancers than in normal endometrial tissue [103]. The antiestrogen toremifene is used for the treatment of advanced breast cancer in postmenopausal women. Tamoxifen, also an antiestrogen used extensively to treat breast cancer, has an estrogenic effect on the uterus and it probably responsible for the increased incidence of endometrial cancer observed during tamoxifen therapy [104]. Both tamoxifen and toremifene activate PKC isoforms in a human endometrial tumor model [105]. Tamoxifen can also inhibit PKC in breast cancer cells [107]. Estrogens increase PKC-δ expression in the uterus [106]. Normal human endometrial stromal cells exhibit actin filaments organized into stress fibers throughout the cells [108], a phenotype consistent with stationary cells [109]. Treatment of these cells with tamoxifen disrupted actin filaments [110]. Moderately differentiated human endometrial adenocarcinoma RL95-2 cells exhibit disrupted actin aggregates only near the cell periphery and no organized actin filaments [111]. When these cells are treated with tamoxifen, actin filaments reorganize and cells enlarge [110]. Activation of PKC with sn-dioctanoylglycerol (DiC8) in normal human endometrial stromal cells altered fibronectin localization to resemble the pattern observed when stromal cells are transfected with an SV40 large T antigen and c-Ha ras oncogene. This pattern also resembles the fibronectin pattern observed in human endometrial sarcoma cells [112].

A phorbol ester tumor promoter and epidermal growth factor (EGF) was mitogenic in the endometrial cancer cell line HEC-1-A [113]. The effects of EGF were mediated by the PKC pathway and vice versa. A negative feedback loop involving PKC regulated EGF-induced morphological changes. Activated PKC catalyzed phosphorylation of EGF-R [114, 115] and resulted in decreased affinity of the receptor for EGF which led to rapid physiological reduction of EGF signaling [116]. EGF activates PKC in HEC-1-A cells and is also mitogenic in these cells [113].

In a study using normal human endometrial stromal cells and well differentiated human endometrial adenocarcinoma Ishikawa cells, it was suggested that persistent activation of PKC might lead to overexpression of c-fos and c-jun in some endometrial cancers with an estrogen rich microenvironment [117]. These factors may be associated with transformation or growth potential. In one study, estrogen increased PKC activity and c-fos expression in endometrial stromal cells and in Ishikawa cells. Progesterone decreased c-fos and c-jun expression and PKC activity induced by estradiol in the stromal cells, but not in Ishikawa cells, which persistently overexpressed c-fos and c-jun. TPA also increased c-fos and c-jun expression in these cells. Activation of PKC in the endometrium by estrogen, by the endogenous activator DAG, by drugs such as tarnoxifen, or by environmental toxicants that act like estrogens, or phorbol esters may be a step in the multistep process of tumor development in the endometrium.

PKC isoforms play critical, but varied roles in carcinogenesis often depending on the cell or tissue system examined. Understanding the cell biology of PKC is critical to understanding the role of PKC in cancer therapy. An interlocking, organized matrix system extends from the extracellular matrix, through the cytoskeleton, to the nucleus and governs cell shape and function in a normal cell [148]. Virtually every subcomponent of this interlocking matrix system is disrupted in a cancer cell [148]. In vitro studies indicate that transformed cells possess disrupted F-actin or filamentous actin, while normal, stationary cells possess actin filaments organized into stress fibers [108; 148-151]. Upon transformation, cells lose stress fiber bundles and undergo a concomitant alteration in cell shape, loss of contact inhibition and enhanced tumor-forming potential [152-156]. Alterations in intracellular matrix (cytoskeletal proteins) and extracellular matrix proteins which occur during tumorigenesis lead to altered gene expression, cell-cell commnunication, cell adhesion, and migration.

The myristoylated alanine-rich C-kinase substrate (MARCKS) (reviewed in 157-159) is a major PKC substrate. MARCKS binds calcium/calmodulin and crosslinks F-actin, and both of these activities are regulated by PKC-dependent phosphorylation (160-162). PKC phophorylation of MARCKS inhibited the interaction of MARCKS with actin and calmodulin. When activated or phosphorylated, PKC binds to the plasma membrane and MARCKS moves away from the plasma membrane to the cytoplasm (163). When MARCKS is functional in the plasma membrane, it cross-links actin filaments. Upon translocation to the cytoplasm, MARCKS can no longer cross-link actin filaments.

The presence of organized stress fibers has been correlated with the ability of cells to exert enough traction against the substrate to wrinkle a rubber substrate [164]. When these stress fibers were altered by the tumor promoter TPA, cells possessed reduced contractility as determined by their inability to wrinkle a rubber substrate. TPA activates PKC and induces disruption of actin filaments [9, 165, 166]. The reduced contractility of TPA-treated fibroblasts may help explain the increased invasiveness and loss of anchorage dependence by transformed cells [162].

Signaling pathways regulate cellular events associated with cell motility and invasion [165]. Protein kinases regulate cytoskeletal architecture and cell adhesion [166,167]. Numerous studies indicate that PKC is an important regulator of cytoskeletal function in normal and transformed cells (Reviewed by Keenan and Kelleher, 1998; 167). For example, phorbol esters induce actin condensation [168]. A specific actin-binding motif has been identified that is unique to PKC-ε [169]. PKC-ζ regulates the actin cytoskeleton in lymphocytes [170]. A PKC-ζ-based signaling pathway has been described in the regulation of actin in an interleukin-2-stimulated murine T-cell line [171]. Specifically, the interleukin-2-induced pathway controls actin organization by sequential activation of the GTP-binding protein Rho, PI-3 kinase, and PKC-ζ [171]. Specific calcium-independent PKC isoforms redistribute to putative cytoskeletal locations after anti-CD3/PMA activation [172,173].

Further evidence for the role of PKC in the regulation of the lymphocyte cytoskeleton has come from studies examining the effects of PKC inhibitors that have been shown to inhibit lymphocyte motility [174]. Data from these studies suggest that specific PKC isoforms may therefore have defined targets in the lymphocyte cytoskeleton. PKC and the ADP-ribosylation factor nucleotide exchange factor jointly regulate remodeling of the actin cytoskeleton in HeLa cells [175]. Cytoskeletal reorganization during spreading is controlled by a phospholipase D/PKC-dependent pathway in LM3 mouse mammary adenocarcinoma cells [176].

PKC has a role in regulating cell invasion. Higher levels of endogenous PKC were found in a metastatic clone than in a nonmetastatic clone derived from the T10 fibrosarcoma cell line [177]. PKC is involved in mediating focal adhesion formation and thus the association of microfilaments with the plasma membrane [178, 179]. One specific example is that PKC and the G protein rho are necessary for focal adhesion formation [180]. Treatment of cells with retinoic acid increased cell adhesion and decreased cell invasion. Untreated melanoma cells exhibited a diffuse actin staining pattern with no organized stress filaments. Treatment of melanoma cells with 1 μM all-trans retinoic acid caused numerous stress fibers to appear while inhibiting invasion into Matrigel and increasing cell adhesion to collagen and laminin [181, 182]. Retinoic acid also inhibited invasion of human lung carcinoma cells [183] and inhibited invasion of human mammary carcinoma (MCF-7) cells [184].

Retinoic acid alters PKC. Studies have demonstrated a functional link between PKC and retinoid pathways in normal tissue, the differentiation process, and in reversion of the malignant phenotype. PKC activity was reduced by one-third in soluble and membrane fractions of cells from human non-sun-exposed skin treated with 0.1% retinoic acid cream (Retin-A), suggesting that retinoic acid down-regulated PKC in human skin [185]. PKC is expressed in the peripheral nervous system and is involved in differentiation [186]. When a human neuronal-derived crest-cell line expressing high PKC-α levels was treated with 0.1 μM retinoic acid, PKC-α mRNA was suppressed concomitantly with morphological differentiation of cells. This suggests that suppression of PKC-α mRNA is associated with reversion of the malignant phenotype [186]. PKC-α mRNA decreased as HL-60 cells (human leukemia cells) matured to a neutrophil phenotype in response to retinoic acid [187]. Therefore, the PKC-α isoform is specifically down-regulated during human neutrophil terminal differentiation [187]. Retinol, retinoic acid, and beta carotene in nanomolar concentrations blocked stimulation of PKC by long-chain fatty acids in human colonic tumor cells [188]. Retinoic acid induced a decrease in PKC in lung carcinoma cells and suppressed invasion and metastasis [189].

Retinoic acid induced a reversion of the malignant phenotype in endometrial adenocarcinoma cells probably by binding to and inactivating PKC. Retinoic acid induced endometrial differentiation, evidenced by reorganization of actin filaments, and alterations in PKC-α localization, consistent with PKC inactivation [23]. Treatment of moderately differentiated human endometrial RL95-2 cells with retinoic acid caused actin filaments to reorganize into stress fibers, indicating that retinoic acid can convert the cells to a more differentiated, stationary phenotype. These retinoic acid-induced structural changes also induced functional changes because retinoic acid treatment decreased cell detachment in in vitro detachment assays; one-half as many retinoic acid-treated cells detached as in controls [190]. Retinoic acid treatment of RL95-2 cells caused PKC-α to localize exclusively to the cytoplasm, rather than the membrane and cytoplasm as seen in control cells [23]. This relocalization of PKC-α is consistent with inactivation of PKC-α because activated PKC is generally located in the membrane [1]. More recent studies have shown that all-trans retinoic acid binds directly to and inactivates PKC-α [191]. This may represent a general mechanism by which retinoids regulate PKC activity.

One of the current avenues in cancer research is to find selective anticancer drugs without the cytotoxic side effects associated with conventional cancer chemotherapy. In order to accomplish this, targets that are cancer specific will need to be identified, so that the cancer cells can be targeted while normal cells are spared. The results of experiments using isolated PKC suggested that retinoids can act directly to protect the regulatory domain of PKC from oxidative modification induced by oxidants [204]. The inventor has shown that treatment of less differentiated human endometrial adenocarcinoma cells with pharmacological doses of retinoic acid caused differentiation, concurrent with relocalization of PKC-α exclusively to the cytoplasm, indicating PKC-α inactivation [23]. Retinoids offer a natural way to induce differentiation, and may protect against alterations induced by environmental toxicants and carcinogens. In a recent study of 1081 patients in Northern Italy and Switzerland, beta carotene (the provitamin form of retinoic acid) conferred significant protection against the development of endometrial cancer [205]. Retinoic acid deficiency increases susceptibility to cancer. In a recent study, female mice fed a vitamin A-deficient diet developed cervical cancer [206]. Additionally, in a cross-sectional sampling of 116 women receiving care at a family planning clinic, those with cervical dysplasia had significantly lower plasma levels of beta carotene than women with normal cervical cytology [207].

Various types of cells lines are known, but none have the limitations and characteristics of the CAC-1 cell line disclosed herein. The following patents are arguably relevant to the cell line and methods disclosed herein:

| Number | Inventor(s) | Issue Date |
|---|---|---|
| 1. 5,948,679 | Park et al. | 09/07/99 |
| 2. 5,928,894 | Lal et al. | 07/27/99 |
| 3. 5,914,227 | Nakamura et al. | 06/22/99 |
| 4. 5,858,721 | Naughton et al. | 01/12/99 |
| 5. 5,821,121 | Brothers | 10/13/98 |
| 6. 4,757,005 | Chan | 07/12/88 |
| 7. 5,710,038 | Mes-Masson et al. | 01/20/98 |
| 8. 5,695,996 | Quinn et al. | 12/09/97 |
| 9. 5,643,787 | Barsky et al. | 07/01/97 |
| 10. 5,518,915 | Naughton et al. | 05/21/96 |
| 11. 5,266,480 | Naughton et al. | 11/30/93 |

The following published scientific literature and support materials are arguably relevant to the cell line and methods disclosed herein:

A. Boyd et al., Analysis of Oncogene Alterations in Human Endometrial Carcinoma, Molecular Carcinogenesis 4:189-195 (1991).

B. Richardson et al., KLE: a Cell Line with Defective Estrogen Receptor Derived from Undifferentiated Endometrial Cancer, Gynecologic Oncology 17:213-230 (1984).

C. Way et al., Characterization of a New Human Endometrial Carcinoma (RL-95-2) Established in Tissue Culture, In Vitro, Vol. 19, No. 3 (Part I), March 1983.

D. American Type Culture Collection, catalog listings for materials available as of September 1991: page 154 (for KLE); page 160 (for RL95-2); and page 254 (for ANC3CA and HEC-1-A).

The following scientific papers authored by the inventor (among others) are arguably relevant to the cell line and methods disclosed herein:

Carter, et al., A Newly Characterized Human Endometrial Adenocarcinoma Cell Line (CAC-1) Differentiates in Response to Retinoic Acid Treatment, Expt. and Molecular Pathology, 69: 175-191 (2000).

Carter, Protein Kinase C as a Drug Target: Implications for Drug or Diet Prevention and Treatment of Cancer, Current Drug Targets, 1: 163-183 (2000).

Radominska-Pandya et al., Direct Interaction of All-trans-retinoic Acid with Protein Kinase C: Implications for PKC Signaling and Cancer Therapy, Journal of Biological Chemistry, Vol. 275, 29:22,324-22,330 (2000).

Carter et al., Cytoskeletal Reorganization Induced by Retinoic Acid Treatment of Human Endometrial Adenocarcinoma (RL95-2) Cells is Correlated with Alterations in Protein Kinase C-∝, Pathobiology, 66: 284-292 (1998); incorporated herein by reference.

Carter et al., State of Differentiation Affects the Response of Endometrial Adenocarinoma Cells to Retinoic Acid, Anticancer Research, 17:1973-1984(1997).

Carter et al., Effects of retinoic Acid on Cell Differentiation and Reversion Toward Normal in Human Endometrial Adenocarcinoma (RL95-2) Cells, Anticancer Research, 16: 17-24 (1996).

There are a number of cell lines known in the art, which are distinguishable from CAC-1 cells for various reasons. For a convenient list of most of those cell lines, see Analysis of Oncogene Alterations in Human Endometrial Carcinoma published in Molecular Carcinogenesis 4:189-195 (1991), especially Table 1. Below is a brief listing of the main differences of each cell line. In general, CAC-1 cells differ from other isolated cell lines in that CAC-1 cells are from a Grade III endometrial tumor and from a primary tumor. Other known cell lines are usually more differentiated (Grade II or I), less invasive, and originate from a different cancer tumor or lesion (usually a secondary tumor or lesion rather than a primary tumor, or from a different body tissue). CAC-1 cells also have different karyotypic abnormalities than those disclosed for some of the other known isolated cell lines. The following is a summary of some of the main distinctions between the respective cell line and CAC-1 cells.

RL95-2 cells: Already known in the art is the line of RL95-2 cells, which are endometrial adenocarcinoma cells derived from a Grade II lesion. (See, In Vitro Vol. 19, No. 3 (Part I), March 1983.) These cells typically have an extra 47^{th} chromosome, from being triploid rather than diploid at chromosome 8. (Id.)

KLE cells: These cells were obtained from a metastatic endometrial adenocarcinoma lesion that metastacized to the colon of a patient pretreated with chemotherapy. They appear much larger and more irregular than CAC-1 cells too. KLE cells also grow more slowly than CAC-1 cells. These cells are not as responsive as CAC-1 cells are to retinoic acid treatment.

EA-1 cells: These are mesenchymal cells, possibly fibroblasts, rather than epithelial in origin.

ANC3A cells: These are from a lymph node of a patient with a metastatic lesion of endometrial adenocarcinoma.

HEC-1-A cells: Grade II, moderately differentiated endometrial adenocarcinoma cells.

HOUA cells: Grade II, moderated differentiated adenocarcinoma cells.

HHUA cells: Grade I, well differentiated adenocarcinoma cells.

Ishikawa cells: Grade I, well differentiated endometrial adenocarcinoma cells of the endometrial epithelium of the uterus. They are not responsive to the retinoic acid treatment, as CAC-1 cells are.

### BRIEF SUMMARY OF THE INVENTION

In general, this invention relates to one or more living cells, especially those isolated from humans. This invention also relates to methods of culturing or otherwise growing one or more cells, and methods of using one or more cells.

A primary object of this invention is to provide a cell that multiplies consistently and relatively quickly. Another object is to provide a cell that retains the characteristics of the cell or tissue from which it originated.

One object of the invention is to provide a method of culturing or otherwise reproducing cells that assures the cell proliferation. Another object of the invention is to provide a cell that is useful in investigating cellular functions such as, for example, inhibition of protein kinase C. Yet another object is to provide a method of determining the effect of a protein kinase inhibitor on a condition in a cell, especially one having manifestations consistent with cancer. A similar object includes providing a method for studying the mechanism by which a condition in a cell having manifestations consistent with cancer reverts to a less cancer-like phenotype, or to provide a method for studying the mechanism by which a condition in a cell having manifestations consistent with cancer remains only partly differentiated.

Another object includes providing a method of identifying a compound that enhances the cross-linking between MARCKS and actin filaments. Yet another object includes providing a method of enhancing cross-linking of actin filaments in cell cytoplasm.

A more general object includes providing a method of enhancing cell differentiation.

Another method includes providing a method, using a cell isolated in vitro, for predicting the effect on cell differentiation attributable to a differentiation enhancing test compound to be applied to an in vivo cancer cell.

Other objects will become expressed or apparent in the remainder of this application.

### BRIEF DESCRIPTION OF THE SEVERAL VIEWS OF THE DRAWINGS OR MICROGRAPHS

The following describes the drawings accompanying this application, which are incorporated herein.

FIG. 1A is a micrograph showing a colony of cells initially established in a monolayer, this differential interference contrast image of CAC-1 cells at passage 29 shows that the cells form tightly packed colonies. (450×. Bar=100 μm.)

FIG. 1B is the same colony at higher magnification. The cells at passage 12 appear rounded and form a dense monolayer. (Magnification=1200×; scale bar=50 μm.)

FIG. 1C shows a colony at passage 4, displaying stromal cells (101) encapsulating adenocarcinoma cells (102); the mixed culture was stained for F-actin. The stromal cells that surround the adenocarcinoma cells possess parallel bundles of actin filaments (arrow 103); the population of adenocarcinoma cells, however, possess F-actin only as disrupted aggregates near the cell periphery. (Magnification=1200×; scale bar=50 μm.)

FIG. 1D shows a colony in another area, at passage 4, displaying stromal cells (104) encapsulating adenocarcinoma cells or CAC-1 cells (105); CAC-1 cells (105) are also surrounded by stromal cells that possess organized actin filaments (106) running parallel to the long axis of the cells (arrow 106). The adenocarcinoma cells only display actin as disrupted aggregates (107) near the cell periphery; these cells were immunostained with fluorescein-labeled phalloidin and then with TRITC-labeled vimentin. In this micrograph, only actin is visualized because or the use of an argon laser of 488-nm wavelength and a 520-nm long pass barrier filter. (Magnification=1200×; scale bar=50 μm.)

FIG. 1E shows a colony of cells at passage 29, immunostained for cytokeratin 19; adenocarcinoma cells(108) stain positively for cytokeratin. (Magnification=1200×; scale bar=50 μm.)

FIG. 1F shows stromal cells (109) exhibiting positive vimentin staining, while adenocarcinoma cells (110, to the left of the stromal cells) did not stain positively; this image was collected using the krypton laser of 568-nm wavelength and a 610-nm long pass barrier filter to visualize TRITC-labeled vimentin. (Magnification=1200×; scale bar=50 μm.)

FIGS. 2A through 2G are transmission electron micrographs of CAC-1 cells at passage 10 showing that the cells possess typical epithelial characteristics, with specializations typical of adenocarcinoma cells.

FIG. 2A is a low magnification (2000×) micrograph of cultured cells showing an area of a colony of cells displaying epithelial features such as numerous long and branching microvilli at the cell surface (201), cell margins delineated by well formed junctions, and a well defined terminal web (202) of actin filaments. (Bar=3 μm.)

FIG. 2B is a micrograph at a slightly higher magnification (2500×), showing actively dividing cells. These are apparent due to the presence of mitotic figures (203).Pleomorphic nuclei containing acentric nucleoli (204) are present. (Bar=3 μm.)

FIG. 2C is a micrograph showing some large multi-nucleated cells. (Bar=3 μm.)

FIG. 2D is a micrograph showing intercellular lumens (205), common in the CAC-1 cell line. (Bar=3 μm.)

FIG. 2E is a micrograph at higher magnification, showing the junctional complex of FIG. 2D, and showing a desmosome (206), intermediate junction (207), and a tight junction (208) at the luminal surface. (Bar=0.5 μm.)

FIG. 2F is a high magnification electron micrograph showing a desmosome between adjoining cells (209). (Bar=0.5 μm.)

FIG. 2G is a micrograph showing that below the apical junctional complexes of CAC-1 cells, cell processes closely interdigitate (210) and are joined by desmosomes (211), a pattern characteristic of adenocarcinomas. (Bar=0.5 μm.)

FIG. 3 is a micrograph showing the chromosomes of a typical CAC-1 cell; the cytogenetic results show that the cells have 48 chromosomes (numbered 1 through 22, plus double X), and are hyperdiploid, with complex chromosomal aberrations. There is trisomy of chromosomes 3, 7, and 17.

FIG. 4A is a micrograph showing CAC-1 cells at passage 12, exposed to media alone (i.e., control). Treatments were for 90 min at 37° C. Note F-actin as disrupted aggregates near the cell periphery (arrowheads 401). (1200×. Bar=50 μm).

FIG. 4B is a micrograph showing CAC-1 cells at passage 12, exposed to ethanol (i.e., vehicle control). Treatments were for 90 min at 37° C. Note F-actin as disrupted aggregates near the cell periphery (arrowheads 402). (1200×. Bar=50 μm).

FIG. 4C shows CAC-1 cells at passage 12, treated with 1 μM 13-cis retinoic acid; cells enlarged and actin filaments became apparent transversing the cells; actin filaments reorganize (arrow 403) in the enlarged cells. (1200×. Bar=50 μm).

FIG. 4D shows CAC-1 cells at passage 12, treated with 1 μM all-trans retinoic acid, which induced actin filament reorganization (arrow 404) throughout these enlarged cells. (1200×. Bar=50 μm).

FIG. 5 depicts mean cell size of the cells shown in FIG. 4; cells treated with 1 μM 13-cis retinoic acid or 1 μM all-trans retinoic acid were twice the size of cells only exposed to media or ethanol. Measurements of cell perimeters were performed using proprietary software running on a Zeiss confocal laser scanning microscope; twenty-five cells were measured for each treatment group and the mean and standard error calculated.

FIG. 6 depicts the mean number of detached cells in medium (control), ethanol (vehicle control), 1 μM 13-cis and 1 μM all-trans retinoic acid-treated CAC-1 cells. There was a decrease in cell detachment following retinoic acid treatment. This experiment was performed in duplicate and is consistent with data from two additional experiments. Retinoic acid treatment decreased cell detachment by around 40% compared to controls.

FIG. 7 depicts a growth curve of CAC-1 cells in an 8-day growth period; retinoic acid treatment induced a slight lag in growth of CAC-1 cells. Control, vehicle control and retinoic acid-treated cells were plated onto 60 mm dishes at 10,000 cells/plate and maintained in culture for 12 days. Cells were treated on day 2 and retreated every third day. Cells were trypsinized on days 4, 7, 10 and 12 and counted with a hemacytometer. Numbers represent mean cell numbers from duplicate dishes for each time point from a representative experiment. Treatment with retinoic acid caused a lag in cell growth compared to controls.

FIG. 8A is a micrograph of media treated (control) cells showing plasma membrane staining (arrow 801) for PKC-α, along with diffuse cytoplasmic staining (802). (1200×. Bar=50 μm.)

FIG. 8B shows ethanol treated (vehicle control) cells showing plasma membrane staining (arrow 803) for PKC-α, along with diffuse cytoplasmic staining (804). (1200×. Bar=50 μm.)

FIG. 8C shows the localization of PKC-α in the cytoplasm, following treatment with 1 μM 13-cis retinoic acid treatment; PKC-α staining is localized to the cytoplasm (805) with distinct perinuclear staining (806). (1200×. Bar=50 μm.)

FIG. 8D shows the localization of PKC-α in the cytoplasm, following treatment with 1 μM all-trans retinoic acid treatment, which caused PKC-α to localize to the cytoplasm (807) and perinuclear areas (808). (1200×. Bar=50 μm.)

FIG. 9A is a Western Blot. Analysis of the subcellular distribution of PKC-α in CAC-1 cells by cell fractionation and immunoblotting shows that retinoic acid treatment caused a decrease in the amount of PKC-α in the particulate fraction and a slight increase in the amount of PKC-α in the cytosolic fraction. Cytosolic and particulate fractions were prepared from CAC-1 cells and 30 μg of protein/lane was applied to SDS/PAGE and PKC-α detected by immunoblotting. Lanes 1 and 2 show cytosolic and particulate fractions of media control cells, respectively; Lanes 3 and 4 show cytosolic and particulate fractions of cells treated with 1 μM all-trans retinoic acid, respectively; Lanes 5 and 6 show cytosolic and particulate fractions of cells treated with 1 μM 13-cis retinoic acid, respectively; Lanes 7 and 8 show cytosolic and particulate fractions of cells treated with 100 nM TPA (for 20 min), respectively. Lane 9 shows positive control rat brain lysate.

FIG. 9B shows the integrated density values for PKC-α.

FIG. 10A is a micrograph showing CAC-1 cells immunostained for MARCKS, for 90 minutes at 37° C. (the same for all treatments in this series); CAC-1 cells exposed to media only exhibit MARCKS staining (1001) in the cytoplasm, with many cells exhibiting prominent perinuclear staining (arrow 1002). (1200×. Bar=50 μm.)

FIG. 10B is a micrograph showing CAC-1 cells treated with 1 μM all-trans retinoic acid; MARCKS (1003) localized to the plasma membrane. (1200×. Bar=50 μm.)

FIG. 10C is a micrograph showing CAC-1 cells treated with 1 μM 13-cis retinoic acid; MARCKS (1004) localized to the plasma membrane. (1200×. Bar=50 μm.)

FIG. 10D is a micrograph showing the effects of treatment with 14 nM bisindolylmaleimide, a specific inhibitor of PKC, that caused MARCKS (arrowheads 1005) to localize to the plasma membrane; a few cells exhibited cytoplasmic and perinuclear staining. (1200×. Bar=50 μm.)

FIG. 11 depicts a schematic representation of one possible mechanism of retinoid-induced differentiation of CAC-1 cells. In untreated cells, PKC is localized to the plasma membrane and scattered diffusely throughout the cytoplasm. MARCKS is diffusely scattered in the cytoplasm and often localized to perinuclear regions. F-actin only occurs as disrupted aggregates near the plasma membrane. Upon retinoic acid treatment, PKC moves away from the plasma membrane to a perinuclear localization and MARCKS localizes to the plasma membrane where it can cross-link actin filaments.

### DETAILED DESCRIPTION OF THE INVENTION

The invention is essentially a cell line recently isolated from a patient with metastatic adenocarcinoma consistent with primary endometrial cancer. CAC-1 cells originated from glandular epithelial cells forming a primary tumor of the endometrium of the uterus. In general, these cells are poorly differentiated and highly invasive. Besides manifestations of abnormal karyotype, another distinguishing characteristic may be that typical CAC-1 cells are hyperdiploid, having 48 chromosomes rather than the standard 46 chromosomes; they are triploid at chromosomes 3, 7 and 17, but only haploid at chromosome 14.

CAC-1 cells grow relatively well in culture, dividing rapidly and generating colonies having volume relatively quickly, after fewer passages than other known cell lines. Retinoic acid treatment results in cell differentiation relatively quickly, as evidenced by cell enlargement and reorganization of previously-disorganized actin filaments. The invention may also include the method of making (culturing) the CAC-1 cells, as set forth herein. The invention may also include use of the cells, especially in connection with retinoic acid treatment. Such uses may be helpful in research such as, for example, differentiation and invasion studies. CAC-1 cells may also be useful in various assays or other methods for diagnosis or predicting the effectiveness of proposed treatment of diseases or conditions manifested by reduced cell differentiation, especially endometrial cancer. The invention includes a line of cells originating from a specimen of human endometrial adenocarcinoma wherein a majority of said cells behaves in substantially equivalent ways at the morphological, physiological and biochemical levels as a cell of said sample.

For the sake of simplicity and to give the claims of this patent application the broadest interpretation and construction possible, the following definitions will apply:

1. The word “differentiation” means changes in morphology, enzyme activity or protein composition; cellular differentiation that may represent a qualitative change in phenotype that is the consequence of changes in gene expression, ultimately leading to a specific functional competence.

2. The phrase “poorly differentiated” means characterized by a cancer that is grade III, especially endometrial cancer that has spread beyond or outside the uterus, but remains confined to the pelvic area. (Poorly differentiated adenocarcinomas have been clearly recognized as a group of lesions with a high incidence of regional lymph node involvement.)

3. The phrase “retinoic acid treatment” means the treatment(s) described herein including the use one or more retinoic acid(s), especially the 1 μM 13-cis and the 1 μM all-trans versions disclosed herein; it may also mean treatment(s) which, although not fully expressly described herein, are comparable as far as the ability to induce differentiation such as (for example) as evidenced by actin filament reorganization and cell enlargement.

4. The phrase “substantial plurality” means more than merely de minimis, but not necessarily almost a majority.

Also for the sake of simplicity, the conjunctive “and” may also be taken to include the disjunctive “or,” and vice versa, whenever necessary to give the claims of this patent application the broadest interpretation and construction possible. Likewise, when the plural form is used, it may be taken to include the singular form, and vice versa.

In its most general form, the invention includes cellular composition comprising cells isolated from a poorly differentiated uterine cancer. Said cancer may include endometrial cancer; said cells may originate from epithelial cells, particularly glandular epithelial cells.

In one version of the invention, said cancer comprises adenocarcinoma. More particularly, said cancer may have all or substantial characteristics consistent with a primary tumor, especially of adenocarcinoma. One version also includes the characteristic of being metastatic.

A preferred embodiment includes a cellular composition wherein a majority (or possibly a substantial plurality) of said cells comprise a cell line characterized as poorly differentiated. One characteristic of one version of the invention is that a plurality (and possibly a majority) of said cells have at least 48 chromosomes. More particularly, a plurality (and possibly a majority) of said cells are at least triploid at chromosomes 3, 7 and 17, but only haploid at chromosome 14. An even more specific version of the invention includes a cell line wherein a plurality (and possibly a majority) of said cells have at least the following karyotypic characteristics: 48, XX, ?t (1:20) (p?34.3; p11.2), dup (2) (q11.1q23), +3, del (5) (q?23q?31), ?add(6) (p23), add (7), (p?21), +add (7) (q22), der(9;14), (q10;q10), add (15) (p11), +der (17) t(17,;19) (p11.1;p11.1), I (19) (q10), ?del (20) (p?11.2).

The invention may also include a method of culturing, or otherwise growing, said cells. One version includes a cellular composition wherein said cells are grown in vitro as a monolayer.

One version of the invented method includes a method of culturing cells in vitro, comprising the steps introducing said cells to a growth media comprising a 1:1 mixture of Medium 199 and Ham's F12 supplemented with up to 4% serum, antibiotics, anti-micotics and growth factors; and culturing said cells under conditions for proliferation.

Another more particular version of the invention includes a method of culturing cells comprising the steps of:

a. Suspending tissue, separated from a specimen cancer tissue, in a digestion media including (comprising) a mixture of collagenase A in a growth media comprising Medium 199 and Ham's F12 supplemented with fetal bovine serum, bovine calf serum, penicillin streptomycin, L-glutamine, fungizone and insulin transferrin selenium,

b. centrifuging said suspension until pellet formation,

c. introducing said pellet to a growth media including media comprising Medium 199 and Ham's F12 supplemented with fetal bovine serum, bovine calf serum, penicillin streptomycin, L-glutamine, fungizone and insulin transferrin selenium,

d. centrifuging said introductory solution, and

e. plating a supernatant onto a plating dish including culture media comprising Medium 199 and Ham's F12 supplemented with fetal bovine serum, bovine calf serum, penicillin streptomycin, L-glutamine, fungizone and insulin transferrin selenium.

An even more specific version of the invented method includes the steps of:

a. obtaining a hysterectomy specimen of endometrial adenocarcinoma, and placing said specimen in media 1 including Leibovitz L-15 media, 5% penicillin/streptomycin, 500 μg/ml gentamicin and 2.5 μg/ml fungizone;

b. washing said specimen with Hank's Balanced Salt Solution (“HBSS”);

c. mincing said specimen into 1 mm pieces, with sterile blades;

d. washing said pieces with HBSS;

e. centrifuging said pieces for 3 minutes at 700 rpm, until pellet formation;

f. exposing said pellet to 2 μg/ml of collagenase A in growth media 2 comprising a 1:1 mixture of Medium 199 and Ham's F12 supplemented with 1% fetal bovine serum, 3% bovine calf serum, 5% penicillin streptomycin, 4 mM L-glutamine, 2.5 μg/ml fungizone and 0.1% insulin transferrin selenium, for 1 hr at 37° C., pipetting vigorously every 15 minutes;

g. centrifuging at 700 rpm for 3 minutes, resulting formation of a pellet and a first supernatant;

h. plating said first supernatant onto at least one 60 mm plating dish including growth media 2;

i. re-exposing said pellet to said collegenase A in said growth media 2, for 1 hr at 37° C., pipetting vigorously every 15 minutes, resulting in formation of a second supernatant;

j. plating said second supernatant onto at least one 60 mm plating dish including growth media 2, for growth of a plurality of cells;

k. following approximately one month in said culture, and using a sterile pipette under sterile conditions, gently scraping a plurality of cells from said dish and re-plating said cells onto at least one 60 mm plating dish including growth media 2, for growth of a plurality of cells;

l . after approximately 1 week in said culture, transferring a plurality of cells to at least one 100 mm plating dish including growth media 2, and maintaining said cells in said media until cells become essentially confluent therein; and

m. approximately every 3 to 4 days, splitting the cells of said dish into fractions, then transferring each fraction of same into one of a plurality (approximately 4) of plating dishes including growth media 2, and maintaining said cells in said media until formation of additional cells, repeating as many times as desired.

Besides the culturing methods described herein, the invention may also include various methods of using the cells. For example, the invention may include a method of identifying a compound that inhibits the activity of a protein kinase in a cell, comprising the steps of providing a cultured cell, contacting said cell with at least one inhibitor test compound, and determining whether a protein kinase primarily localizes away from the cell membrane. Said localization may be an indication that said test compound likely inhibits said protein kinase. A more particular version of this invention includes a method wherein said protein kinase is an isoform known to be involved in hindering the organization of cytoskeleton matrix in the cell cytoplasm, and determining whether said isoform localizes primarily away from the cell membrane. A more specific version includes a method wherein said protein kinase is protein kinase C, specifically PKC-α, and said inhibitor test compound is retinoic acid; the final step is also determining whether PKC-α localizes primarily in a perinuclear region.

The invention disclosed herein may also include a method of determining the effect of a protein kinase inhibitor on a condition in a cell having manifestations consistent with cancer. Said method includes the the steps of providing a cultured cell, contacting said cell with at least one inhibitor of protein kinase known to be present in abnormally high levels in cells failing to undergo organization of cytoskeleton matrix in the cell cytoplasm, and determining whether protein kinase primarily localizes away from the cell membrane. Said localization may be an indication that said cell is apt to undergo organization of cytoskeleton matrix in the cell cytoplasm. Another more particular version includes a method wherein said protein kinase is PKC-α and said inhibitor of protein kinase is retinoic acid; the final step is determining whether PKC-α localizes primarily in a cytoplasmic and perinuclear region.

Besides the aforementioned methods, the invention disclosed herein includes a method for studying the mechanism by which a condition in a cell, having manifestations consistent with cancer, reverts to a less cancer-like phenotype. This method includes the steps of providing a cultured cell, contacting said cell with at least one inhibitor of protein kinase known to be present in abnormally high levels in cells failing to undergo organization of cytoskeleton matrix in the cytoplasm, and determining whether protein kinase primarily localizes away from the cell membrane. Said localization may be an indication that said cell is apt to undergo organization of cytoskeleton matrix in the cytoplasm. Another more particular version of the invention includes a method said protein kinase is PKC-α and said inhibitor of protein kinase is retinoic acid, and determining whether PKC-α localizes primarily in a cytoplasmic and perinuclear region. The same methods may also be advantageous for studying the mechanism by which a condition in a cell having manifestations consistent with cancer remains only partly differentiated.

Another method includes a method of identifying a compound that enhances the cross-linking between MARCKS and actin filaments in cell cytoplasm, comprising the steps of providing a cultured cell, contacting said cell with at least one enhancer test compound, and determining whether actin filaments organize a cytoskeleton matrix. Said organization may be an indication that said test compound likely allows MARCKS to cross-link actin filaments. A more particular version of the invention includes a method wherein said enhancer test compound is retinoic acid. A more specific version includes a method wherein said enhancer test compound is retinoic acid and said protein kinase is PKC-α, and determining whether PKC-α localizes primarily in cytoplasmic and perinuclear regions. The same methods may also be advantageous for enhancing cross-linking of actin filaments in cell cytoplasm.

Another method includes a method of enhancing cell differentiation, comprising the steps providing a cultured cell, contacting said cell with at least one enhancer test compound, and determining whether actin filaments organize a cytoskeleton matrix. Said organization may be an indication that said test compound likely enhances cell differentiation. A more particular version of the invention includes a method wherein said enhancer test compound is retinoic acid.

There is another method, using a cell isolated in vitro, for predicting the effect on cell differentiation attributable to a differentiation enhancing test compound to be applied to an in vivo cancer cell. This method includes the steps of providing a cultured cell, contacting said cell with at least one enhancer test compound, and determining whether actin filaments organize a cytoskeleton matrix. Said organization may be an indication that said test compound likely enhances cell differentiation. A more particular version of the invention includes a method wherein said enhancer test compound is retinoic acid.

The following is an example of a detailed manner of making CAC-1 cells, and their usefulness.

### EXAMPLE 1

### Introduction

The role of retinoids as differentiating agents has been under investigation for around 2 decades and their use in the diet or clinic remains a promising therapy for the prevention of several types of cancer. Endometrial cancer is the most common form of gynecologic cancer in the United States and the fourth most common cancer in women. In 2000, an estimated 36,100 women in the United States will be diagnosed with endometrial cancer. All-trans retinoic acid combined with tamoxifen is being used in clinical trials to treat advanced breast cancer (Budd, et al., 1998) and 13-cis retinoic acid combined with a-interferon has been used to treat metastatic endometrial cancer (Kudelka, et al., 1993). (Note that the citations to all references in this Example 1 are listed at this end of this section.) Although it has been known for many years that retinoic acid is required for normal differentiation of reproductive epithelia, the role of retinoic acid in endometrial differentiation remains poorly understood. State of differentiation affects prognosis in endometrial cancer. Patients with well-differentiated (Grade I) tumors have 5-year survival rates as high as 87% while patients with poorly differentiated endometrial cancers (Grade III), have 5-year survival rates of only 58%, often with regional lymph node involvement and distant metastasis (Tornus and Elvio, 1993). Differentiation induction by naturally occurring retinoids appears particularly promising for the treatment of endometrial adenocarcinoma especially in postmenopausal women that may be too sick to withstand harsh therapy or surgery. Understanding the mechanisms behind which retinoids exert their differentiating effects is critical to utilizing retinoids as therapeutic agents for this disease.

Previous work by the inventor using human endometrial adenocarcinoma cells at three different stages of differentiation showed that retinoic acid exerted dramatic differentiating effects on moderately differentiated human endometrial RL95-2 cells evidenced by reorganization of actin filaments (Carter and Parham, 1997). The cell lines used for this previous study were originally obtained from different patients with endometrial adenocarcinoma at three stages of differentiation: (1) KLE cells are poorly differentiated (Richardson, et al., 1984), (2) RL95-2 cells are moderately differentiated (Way, et al., 1983), and (3) Ishikawa cells are well differentiated cells (Holinka, et al., 1986). The KLE cells differentiated less than the RL95-2 cells in response to retinoic acid, while the Ishikawa cells were already differentiated and therefore did not exhibit changes in response to retinoic acid treatment. KLE cells were originally derived from a patient with an endometrial metastatic lesion to the colon (Richardson, et al., 1984). Because we needed a better model of poorly differentiated human endometrial adenocarcinoma cells derived from the primary tumor, we established a new cell line termed “CAC-1 cells” in culture. This allowed the investigation of the effects of retinoic acid on differentiation induction in low passage, poorly differentiated human endometrial adenocarcinoma cells.

An organized cytoskeleton is imperative for proper function of cells and tissues (Pienta, et al., 1989). Cytoskeletal organization is manifested by an architecturally organized matrix. Cells with a stationary, differentiated phenotype possess an organized matrix system with organized actin filaments. Virtually every subcomponent of this interlocking matrix system is disrupted in a cancer cell (Pienta, et al., 1989). Transformed cells lose organized actin filaments and undergo a concomitant alteration in cell shape, loss of contact inhibition and enhanced tumor-forming potential (Pienta, et al., 1989; Carter et al., 1994). Forced reorganization of cytoskeletal proteins such as actin can reverse the malignant phenotype. An organized cytoskeleton and increased cell size are associated with differentiation (Rao, et al., 1990). Differentiated cultured cells have markedly higher F-actin levels than undifferentiated cells; specifically, retinoic acid-induced differentiation of HL-60 cells increased their F-actin content 41% above undifferentiated (control) cells and 86-96% above controls after differentiation of human myeloid cells (Rao, et al., 1990). The tumor promotor 12-O tetradecanoylphorbol-13-acetate (TPA) inhibited differentiation of hematopoietic cells and resulted in a 32% decrease in their mean F-actin content (Rao, et al., 1990).

Concomitant with the retinoid-induced reorganization of actin filaments observed previously in RL95-2 cells, retinoid treatment caused PKC-α to localize exclusively to the cytoplasm, indicating inactivation of PKC during differentiation (Carter, et al., 1998). Evidence suggests that the members of the PKC family are activated in specific intracellular compartments by different membrane lipid metabolites, and that PKC isoforms play distinct roles in the control of major cellular functions. PKC is an important regulator of the organization of cytoskeletal and extracellular matrix proteins. Coordinated regulation of PKC activation is crucial for normal cell functions. Increased PKC activity or a change in its subcellular localization may be related to neoplastic transformation (Nishizuka, 1989). Blocking activated PKC may be particularly important for endometrial cancer because PKC activity was significantly higher in endometrial cancers than in normal endometrial tissue (Fujimoto, et al., 1995).

PKC mediates phosphorylation of numerous protein substrates and phosphorylation plays an essential role in regulating PKCs. With the production of diacylglycerol and calcium (for conventional isoforms), PKC is activated and recruited to membranes (Nishizuka, 1988). The myristoylated alanine-rich C-kinase substrate (MARCKS) (Aderem, 1992) is a major PKC substrate. MARCKS crosslinks F-actin and binds calcium/calmodulin; both of these activities are regulated by PKC-dependent phosphorylation. PKC phophorylation of MARCKS inhibits the interaction of MARCKS with actin and calmodulin. When activated or phosphorylated, PKC binds to the plasma membrane, and MARCKS becomes phosphorylated and translocates from the plasma membrane to the cytoplasm, and can no longer cross-link actin filaments (Thelen, et al., 1991). Conversely, when PKC is inactivated MARCKS becomes dephosphorylated and moves to the plasma membrane, where it binds to the sides of actin filaments and cross-links them.

The inventor has established a new cell line of Grade III, poorly differentiated, human endometrial adenocarcinoma cells in culture. (Carter and Madden, 2000.) To the knowledge of the inventor, these CAC-1 cells are the only established cell line of poorly differentiated human endometrial adenocarcinoma cells derived from the primary tumor rather than a secondary tumor or metastatic lesion. These cells have been characterized as epithelial and hyperdiploid, and respond to retinoic acid in a manner similar to other human endometrial adenocarcinoma cells derived from a Grade II lesion, RL95-2 cells (Carter, et al., 1996; Carter and Parham, 1997). The inventor has further investigated the retinoid effects on differentiation of CAC-1 cells, and shown that retinoid treatment induces actin filament reorganization concurrent with changes in PKC-α localization consistent with inactivation of PKC, concomitant with MARCKS relocalization to the plasma membrane. CAC-1 cells represent an ideal model for the investigation of the differentiating effects of retinoids on poorly differentiated human endometrial cells.

### Materials and Methods

### 1. Establishment of Cells in Culture

Tissue was obtained from a hysterectomy specimen from a patient with Grade III human endometrial adenocarcinoma. The patient had not received prior chemotherapy or hormone replacement therapy. The tissue was transported to the laboratory in media including supplements from Gibco, BRL, Gaithersburg, Md. consisting of Leibovitz L-15 media supplemented with 5% penicillin/streptomycin, 500 μg/ml gentamicin and 2.5 μg/ml fungizone. Following a wash with Hank's Balanced Salt Solution (HBSS) (Gibco, BRL), tissue was minced into 1 mm pieces with sterile blades and washed with HBSS. Minced tissue was transferred to a conical tube and centrifuged for 3 min at 700 rpm. The tissue in the resultant pellet was digested using 2 μg/ml of collagenase A (Boehringer Mannheim, Indianapolis, Ind.) in media that included a 1:1 mixture of Medium 199 (Gibco, BRL) and Ham's F12 (Gibco, BRL) supplemented with 1% fetal bovine serum (Hyclone, Logan, Utah), 3% bovine calf serum (Hyclone), 5% penicillin streptomycin, 4 mM L-glutamine (Gibco, BRL), 2.5 μg/ml fungizone and 0.1% insulin transferrin selenium (Collaborative Biomedical Products, Bedford, Mass.) for 1 hr at 37° C. During the 1 hr incubation, the digesting tissue was removed from the incubator and pipetted vigorously every 15 minutes to mechanically disrupt the tissue. When tissue appeared broken apart it was centrifuged at 700 rpm for 3 minutes. The first supernatant was plated. The re-suspended pellet was again exposed to collegenase A and incubated for 1 hr at 37° C. with periodic removal and pipetting as before. The resultant supernatant was plated. This process continued until cells were in suspension.

After one month in culture, cells formed several colonies and the stromal cells had mostly disappeared. Using a sterile small pipette under sterile conditions, some of the larger colonies were gently scraped from the 60 mm dishes and plated onto new 60 mm dishes.

After 1 week, these colonies were confluent and cells were transferred to 100 mm dishes. They were subsequently maintained in these dishes, in media described above, for cell isolation and split at 1:4 ratio every 3-4 days. Cells were checked routinely for mycoplasm using a kit (Sigma, St. Louis, Mo.) and found to be negative.

### 2. Characterization of Cells as Epithelial

Differential interference contrast of cells: Some cells were plated onto 60 mm dishes and allowed to become 70-80% confluent. Differential interference contrast (DIC) images were obtained using a Zeiss confocal laser scanning microscope. Hard copy images were collected on 35 mm Technical Pan film (Kodak) using a Polaroid freeze frame video recorder.

Actin and vimentin staining: Cells at passage 4 were plated onto 4-well Lab-Tek chamber slides (Miles Scientific, Naperville Ill.) at a density of 20,000 cells per well and allowed to attach for 5-7 days. Subsequently cells were rinsed with phosphate-buffered saline (PBS), fixed in 3.7% formaldehyde diluted in PBS for 10 min at room temperature and permeabilized for 5 min with acetone at −20° C. Then cells were incubated with fluorescein isothiocyanate (FITC)-conjugated phalloidin (Molecular Probes, Eugene, Oreg.) at a 1:20 dilution for 20 min at room temperature, and rinsed 3 times with PBS. Some of these cells were additionally stained for vimentin. These cells were incubated with anti-vimentin (Sigma) at a 1:40 dilution for 1 hr at 37° C. Following 2 rinses with PBS, cells were exposed to diluted tetramethylrhodamine isothiocyanate (TRITC)-labelled sheep anti-mouse IgG (Sigma) for 30 min at 37° C. The single or double labeled cells were rinsed and mounted in medium described previously (Carter, et al., 1991) consisting of 5% n-propyl gallate and 0.25% diazobicyclooctane in polyvinyl alcohol and observed with a Zeiss confocal laser scanning microscope. Confocal fluorescent images were obtained using an argon laser of 488-nm wavelength and a krypton laser of 568-nm wavelength combined with appropriate barrier filters. Hard copy images were produced by a Polaroid freeze frame video printer.

Cytokeratin stain: After cells were further established in culture they were stained for cytokeratin, because we hypothesized that the cells were epithelial. CAC-1 cells at passage 29 were plated, fixed and permeabilized as described for actin staining and incubated with a 1:400 dilution of a monoclonal anti-cytokeratin antibody against peptide 18 (Sigma) for 1 hr at 37° C. Following 2 rinses with PBS cells were exposed to a 1:150 dilution of FITC-conjugated sheep anti-mouse IgG (Sigma) for 30 min at 37° C. Cells were rinsed, mounted and observed as described for actin staining.

Electron Microscopy: Cells at passage 14 were plated onto 4-well plastic Lab-Tek chamber slides at 10,000 cells per well and allowed to attach for 5 days. Cells were rinsed twice with warm (37° C.) serum-free growth media and fixed overnight in warm 3% glutaraldehyde in serum-free media at apH of 7.4. Cells were rinsed with 0.1M sodium cacodylate buffer, pH 7.4, and post fixed in buffered 1% osmium tetroxide for 1 hr at room temperature, dehydrated through a graded series of ethanols, then embedded in Polybed 812 (Polysciences, Inc., Warrington, Pa.). After polymerization, the embedded specimens were cut en face with an Leica Ultracut UCT ultramicrotome at 70 nm, mounted on copper grids, and double stained with uranyl acetate and lead citrate. The sections were examined at 80 kV with a LEO EM-910 transmission electron microscope (LEO Electron Microscopy, Inc. Thomwood, N.Y.) at the magnification indicated.

### 3. Cytogenetics

Chromosome analysis in a microscope was performed on 21 trypsin-Giemsa banded metaphase CAC-1 cell cultures (in-situ method) at the 475 band level of resolution. Twenty cells from 20 independent colonies were evaluated for chromosome counts. These cells were from a minimum of 3 independent culture vessels.

### 4. Treatment With Retinoic Acid and F-actin Staining

CAC-1 cells were seeded onto chamber slides at a density of 20,000 cells per well and allowed to attach for 24-48 hrs. Cells were left untreated (media controls) or treated with 1 μM all-trans or 13-cis retinoic acid (Sigma) dissolved in ethanol and diluted in culture media or treated with corresponding doses of ethanol alone in culture media (vehicle controls) for 90 min at 37° C. All retinoic acid exposures were performed in subdued light. Cells were stained for F-actin according to the methods described above. Measurement of cells was performed using proprietary software running on the Zeiss confocal microscope. The area of 25 cells was measured for each treatment group and the mean and standard error calculated.

### 5. Detachment Assays

CAC-1 cells were plated onto 35 mm plastic tissue culture dishes at 100,000 cells per dish and allowed to attach for 7 days. For analysis of the retinoic acid effects on cell attachment, cell detachment assays were performed using the methods of Guo, et al., 1995. Cells were exposed to media alone (controls), 1 μM 13-cis retinoic acid, 1 M all-trans retinoic acid or corresponding doses of ethanol alone in culture media alone (vehicle control) for 24 hrs at 37° C. prior to cell counting. The assays were performed in duplicate. Cells were rinsed twice with sterile HBSS and exposed to 0.5% trypsin/EDTA (Gibco, BRL) for 10 min with constant agitation on a rocker set at 120 rpm's. The media containing the detached cells was collected. Detached cells from each dish were counted using a hemacytometer and the mean calculated and graphed using Cricket Graph. Statistics were performed using StatMost, (DataMost Corporation, Sandy, Utah). Statistical analysis was performed using one way analysis of variance-(ANOVA).

### 6. Growth Curve

Cells were plated onto 60-mm tissue culture dishes at an initial density of 10,000 cells per dish. The assay was performed in duplicate. The following day cells were switched to fresh media (controls), treated with 1 μM all-trans or 1 μM 13-cis retinoic acid or corresponding doses of ethanol (vehicle control). Medium containing fresh retinoic acid was changed every 3 days. On days 4, 6, 10 and 12 of culture, cells were removed from the dishes by trypsinization and counted with a hemacytometer.

### 7. PKC-α and MARCKS Immunostaining

RL95-2 cells were plated onto 4-well Lab-Tek chamber slides at 10,000 cells per well and allowed to attach for 2-4 days prior to retinoic acid treatment. Cells were treated as described for the growth culve for 90 min at 37° C. Prior to MARCKS staining some cells were treated with 14 nM bisindolylmaleimide (Calbiochem, La Jolla, Calif.) for 90 min at 37° C. in order to down-regulate PKC isoforms. Cells were immunostained for PKC-α using an immunofluorescent assay kit and instructions from Research and Diagnostics (Berkley, Calif.) as described previously by Carter, et al., 1998. For MARCKS staining, cells were fixed and permeabilized as described for F-actin visualization and exposed to an anti-MARCKS mouse monoclonal IgG antibody (Upstate Biotechnology, Lake Placid, N.Y.) diluted 1:400 in 1% Bovine Serum Albumin and 0.5% nonfat dry milk (Carnation) in PBS for 1 hr at 37° C. Following 2 rinses with PBS cells were exposed to a 1:150 dilution of FITC-conjugated sheep anti-mouse IgG (Sigma) for 30 min at 37° C. Cells were rinsed, mounted and observed as described for actin staining.

### 8. PKC-α Western

Confluent 100 mm dishes of CAC-1 cells in the exponential phase of growth (70-80% maximum density) were treated for 90 min with media alone or 1 μM doses of all-trans or 13-cis retinoic acid, corresponding amounts of ethanol (vehcile control) or with TPA for 20 min to activate PKC-α. Cytosolic and particulate fractions of cells were isolated as described previously (Carter, et al., 1998) and changes in PKC-α were analyzed by Western blot analysis using methods described previously and a primary antibody from Transduction Laboratories (Carter, et al., 1998).

### Results

### 1. Cell Morphology and Characterization as Epithelial Cells

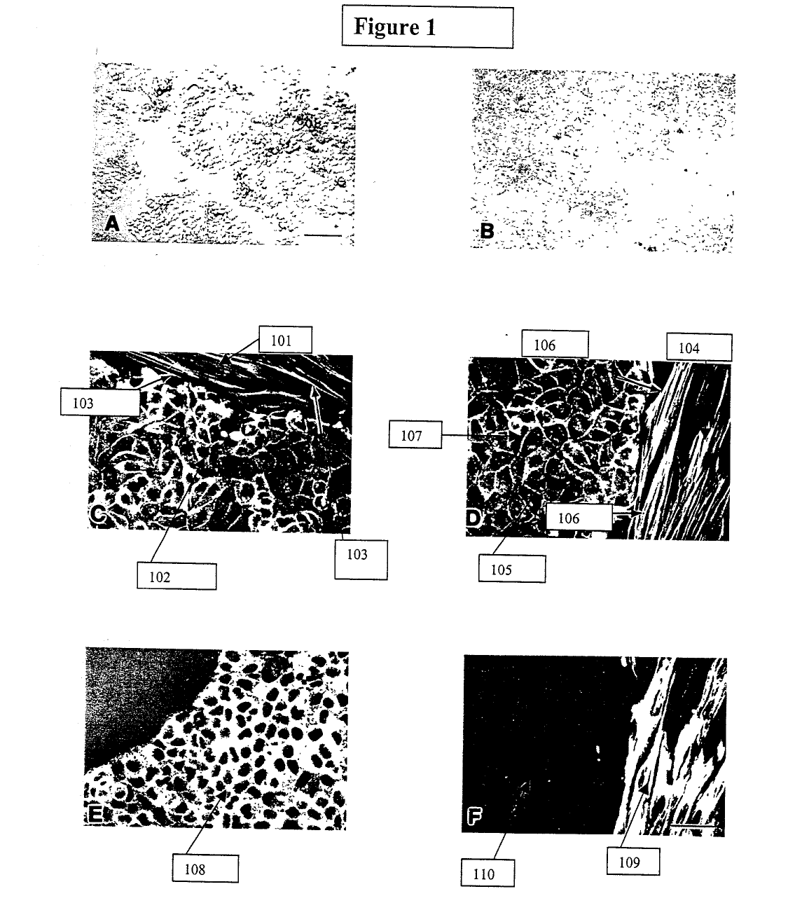

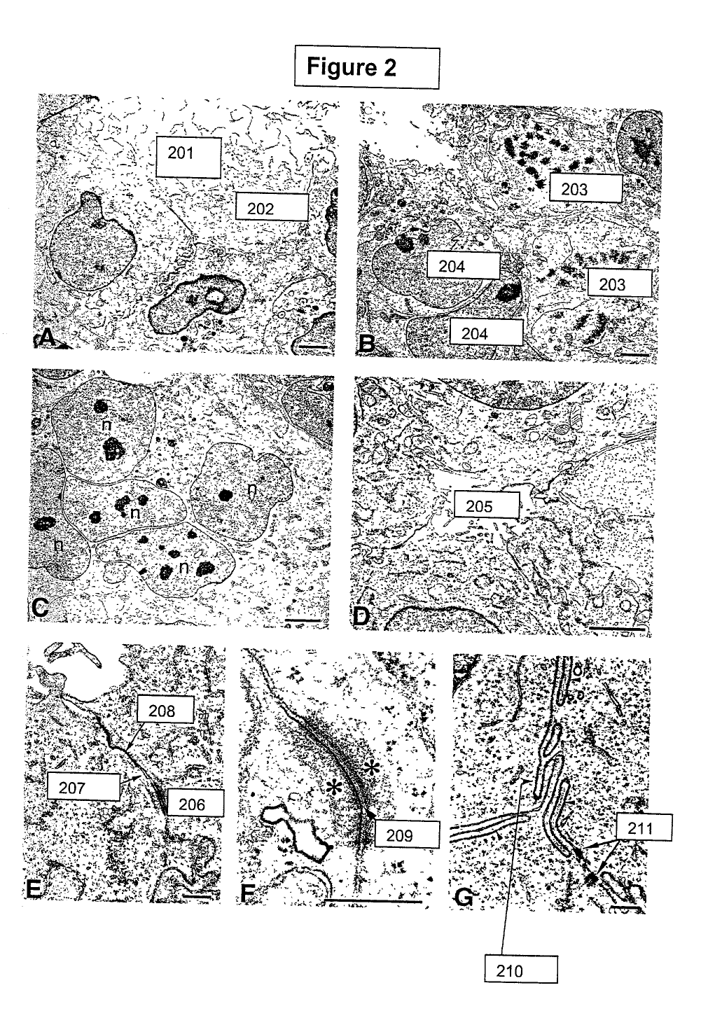

The initial outgrowth of cells resulted in the establishment of several colonies composed of epithelial cells in a monolayer. The largest of these colonies were transferred to new sterile 60-mm dishes. The selected population of epithelial cells was maintained in 60-mm dishes for one week then transferred to 100-mm dishes due to the increased growth rate. Cells formed small tightly packed colonies and these characteristics were maintained as cells were passaged (FIG. 1A). At higher magnifications, rounded cells forming tightly packed monolayers are obvious (FIG. 1B). Early cultures of cells, (passage 4) displayed stromal cells encapsulating adenocarcinoma cells (FIGS. 1C and D). When these cells were stained for F-actin, the stromal cells possessed organized actin filaments running parallel to the long axis of the cells (FIGS. 1C and D). This actin pattern is typical of stromal cells as described previously (Carter, et al., 1991; Carter, et al., 1992). The adenocarcinona cells only displayed actin aggregates near the cell periphery and did not possess any organized stress fibers (FIGS. 1C and D). This disrupted actin pattern is typical of less differentiated human endometrial adenocarcinoma cells (Carter, et al., 1996; Carter and Parham, 1997). As the cells were passaged the stromal cells died, and the adenocarcinoma cells continued to proliferate. In an attempt to characterize these cells as epithelial, they were immunostained for cytokeratin and vimentin. When a colony of established cells, at passage 29, was immunostained for cytokeratin 19, they exhibited positive staining (FIG. 1E). Adenocarcinoma cells in the coculture, at passage 4, did not stain positively for vimentin, but the stromal cells exhibited positive vimentin staining (FIG. F).

### 2. Electron Microscopy

FIG. 2 shows the fine structure of CAC-1 cells. A region of a colony of cells is shown in FIG. 2A. Cells have epithelial features including numerous long, branching microvilli at the cell surface. Cell junctions are obvious and a well-defined terminal web of actin filaments is also apparent. Mitotic figures are obvious in Figure B and indicate that cells are actively dividing. Pleomorphic nuclei containing acentric nucleoli are apparent. Nuclei are prominent and the nuclear to cytoplasmic ratio is high. Some large multinucleated cells are present as seen in Figure C. Five irregularly-shaped nuclei containing multiple nucleoli are apparent in this cell. Intercellular lumens are common in these cells as depicted in Figure D. Several mitochondria are apparent as well as junctional complexes between cells. Figure E is a higher magnification image of a junctional complex seen in Figure D and shows a desmosome, an intermediate junction or zonula adherens and a tight junction or zonula occludens at the lumenal surface. Several ribosomes are also apparent. A desmosome with dense fibrous plaques is prominent at higher magnification in Figure F. Figure G shows that below the apical junctional complexes of CAC-1 cells, cell processes closely interdigitate and are joined by desmosomes.

### 3. Chromosomal Analysis of the Cells

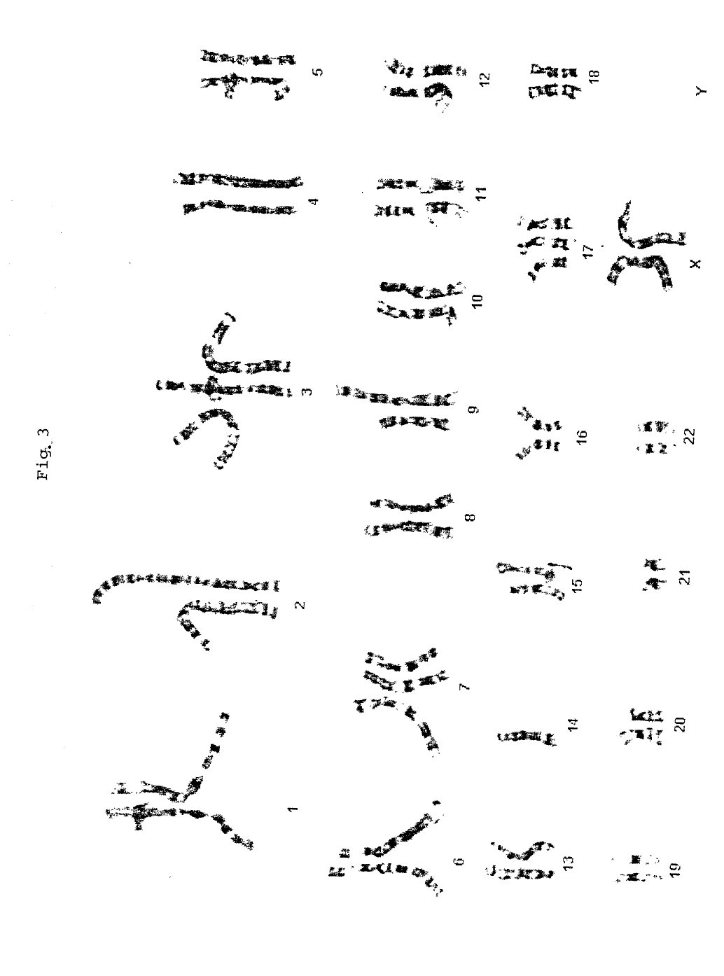

A chromosomal spread is shown in FIG. 3. The cells are hyperdiploid with complex chromosomal abberations. The karyotype of the cells is as follows: 48, XX, ?t (1;20) (p?34.3; p11.2), dup (2) (q11.1q23), +3, del (5) (q?23q?31), ?add(6) (p23), add (7) (p?21), +add (7) (q22), der(9;14) (q10;q10), add (15) (p11), +der (17) t(17;19) (p11.1;p11.1), i (19) (q10), ?del (20) (p?11.2) in 14 cells at passage 27.

### 4. Response to Retinoic Acid

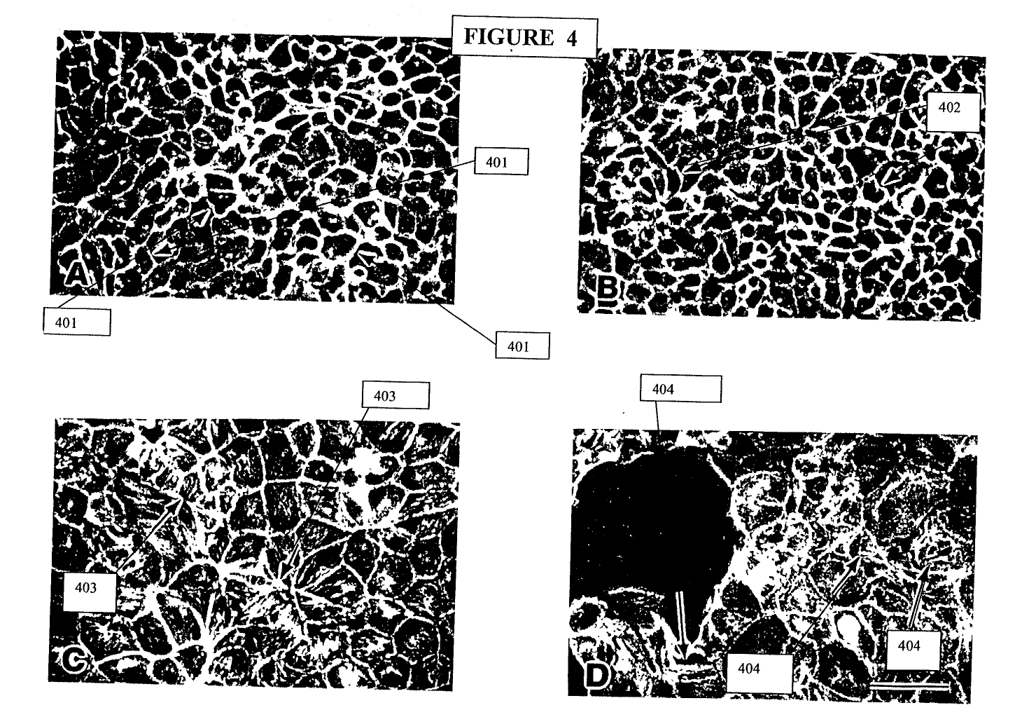

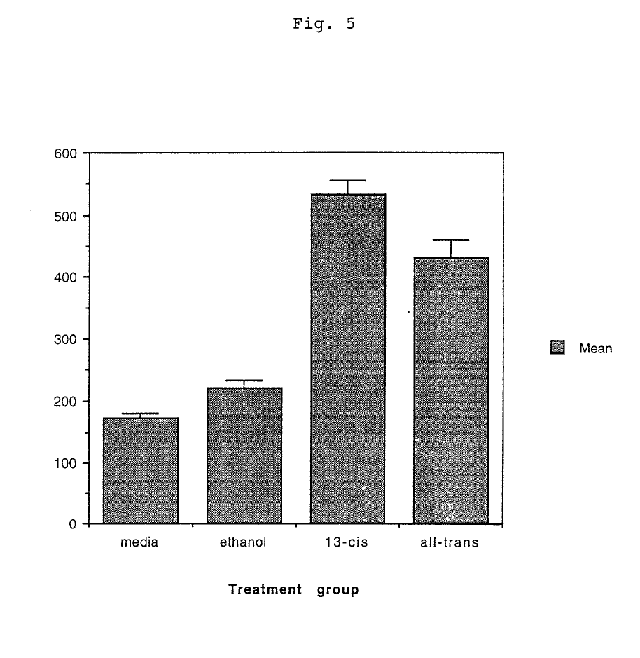

We have previously examined the response of endometrial adenocarcinoma cell lines at various stages of differentiation to the differentiating effects of retinoic acid (Carter, et al., 1996; Carter and Parham, 1997). In this study, we examined the response of the poorly differentiated adenocarcinoma cells or CAC-1 cells to retinoic acid-induced differentiation. Cells, at passage 12, exposed to media alone (controls) or ethanol (vehicle control) and stained for actin, exhibited actin only as aggregates near the cell periphery (FIGS. 4A and B). When cells were treated with 1 μM 13-cis retinoic acid, cells enlarged and actin filaments were apparent (FIG. 4C). Upon treatment with 1 μM all-trans retinoic acid, cells also enlarged and actin filaments were prominent throughout the cells (FIG. 4D). Measurement of cell size showed that cells treated with 13-cis or all-trans retinoic acid were larger than cells only exposed to media or vehicle (ethanol) (FIG. 5). One way analysis of variance (ANOVA), showed that there was no significant difference between the ethanol and medium control cells. Cells treated with retinoic acid were significantly larger than cells exposed to media or ethanol (controls) (P<0.05).

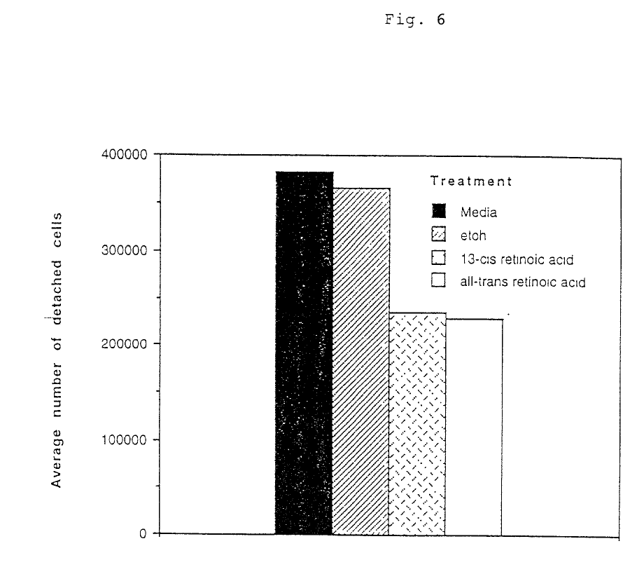

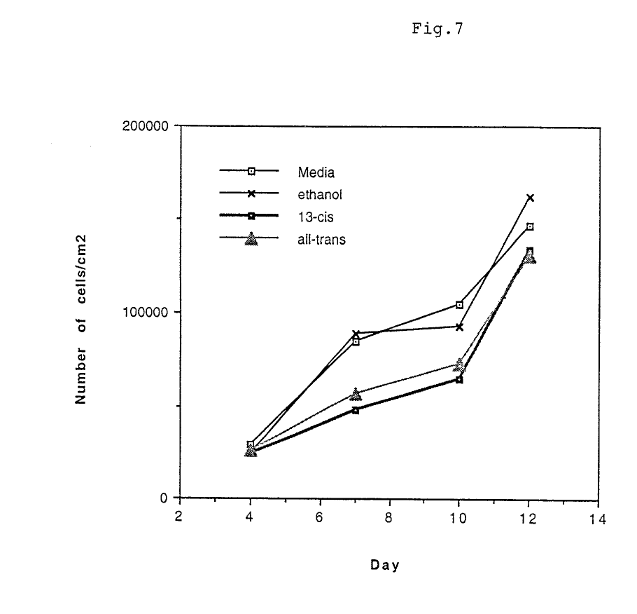

Treatment of CAC-1 cells with retinoic acid increased cell attachment and caused a lag in cell growth. In order to evaluate cell function, cell detachment assays of control and retinoic acid-treated cells were performed based on the methods of Guo, et al., 1995. Cells that have organized actin filaments are consistent with an adherent, stationary phenotype (Pienta, et al., 1989). Cells with disrupted actin are often rounded and poorly adherent (or metastatic). FIG. 6 shows that retinoic acid treatment decreased cell detachment by around 40% relative to control cells. Statistical analysis using ANOVA showed that there was no significant difference between the ethanol and medium control cells. There was, however, a statistically significant difference in the number of detached cells between medium and ethanol control cells compared to both types of retinoic acid treated cells (P<0.05). These data demonstrate that retinoic acid induced a decrease in cell detachment of CAC-1 cells from their substrate compared to controls. This is consistent with the retinoic acid induction of the reorganization of actin filaments in CAC-1 cells. When cell growth was evaluated in an 8 day growth curve, retinoic acid induced a slight lag in cell growth of CAC-1 cells (FIG. 7).

### 5. Effects of Retinoic Acid on Protein Kinase C-α and MARCKS Localization

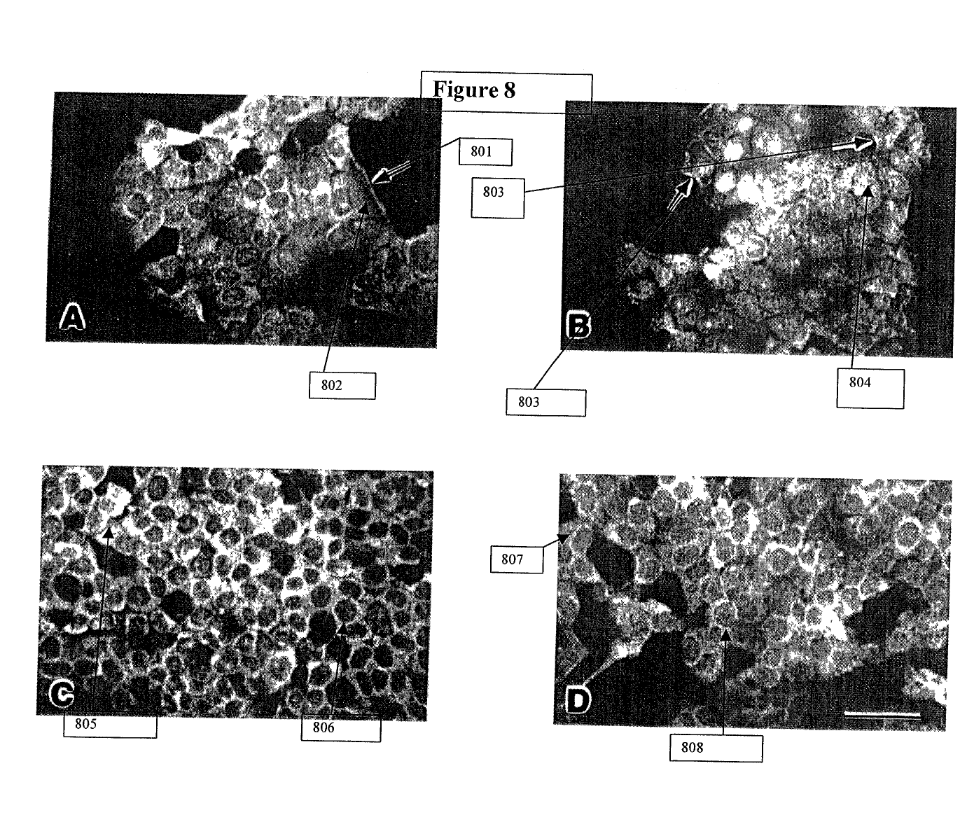

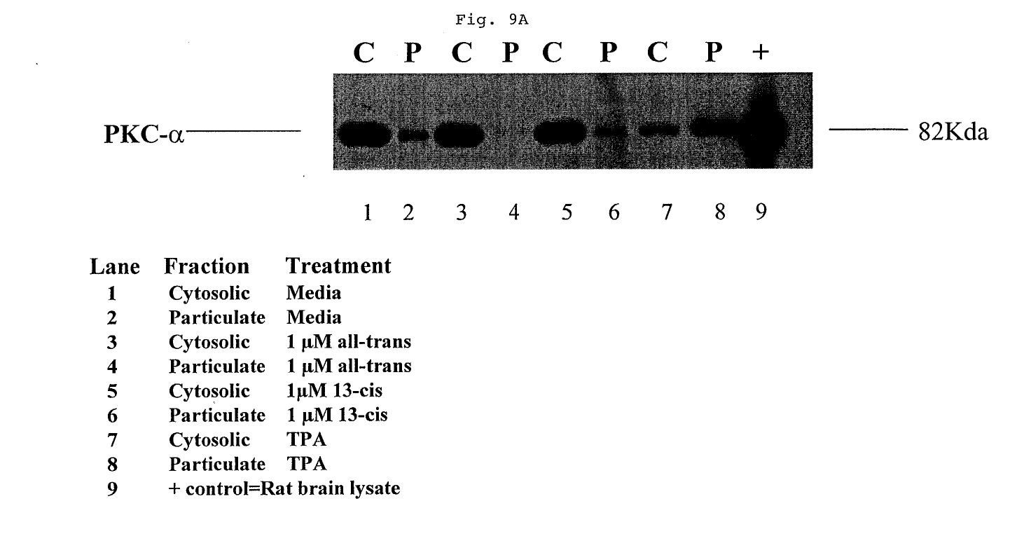

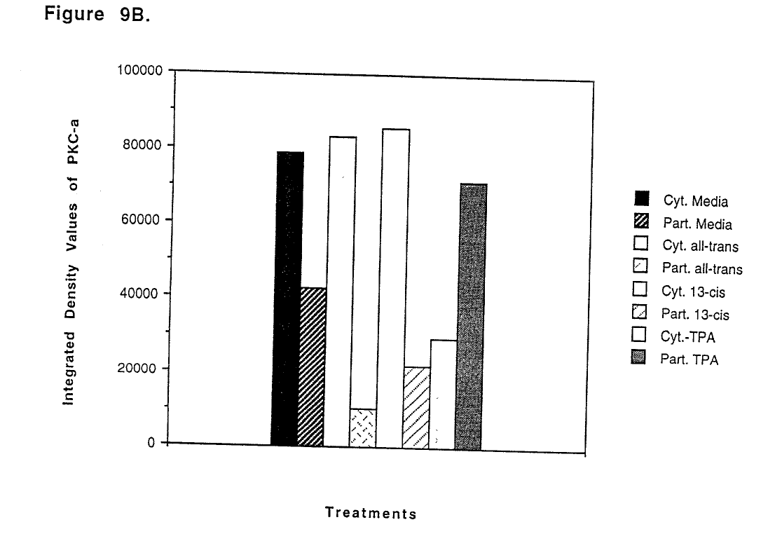

Because we have shown previously (Carter, et al., 1998) that retinoic acid induced reorganization of actin filaments in moderately differentiated human endometrial adenocarcinoma (RL95-2) cells concurrent with a localization of PKC-α to the cytoplasm, we evaluated the effects of retinoic acid on PKC-α subcellular localization in CAC-1 cells. Media control (FIG. 8A) and vehicle (ethanol) control (FIG. 8B) treated cells exhibited PKC-α staining diffusely throughout the cytoplasm along with some plasma membrane staining. Treatment with 1 μM 13-cis (FIG. 8C) or 1 μM all-trans (FIG. 8D) retinoic acid caused PKC-α to localize exclusively to the cytoplasm occurring predominantly in perinuclear regions. Cells that received no primary antibody but only received secondary antibody did not stain. To further document changes in PKC-α localization induced by retinoic acid treatment, particulate and cytosolic protein fractions were examined by immunoblotting (FIG. 9A). The results confirmed those of FIG. 8 and showed a decrease in PKC-α immunostaining in the particulate fraction of cells following retinoid treatment. However, when cells were treated with TPA for 20 min to activate PKC α, most of the PKC-α was present in the particulate fraction. This is the expected outcome since TPA is an activator of PKC and activated PKC is generally located in the plasma membrane (Nishizuka, 1988). Densitometric quantitation also showed that PKC-α was less prevalent in the particulate fraction upon retinoid treatment (FIG. 9B).

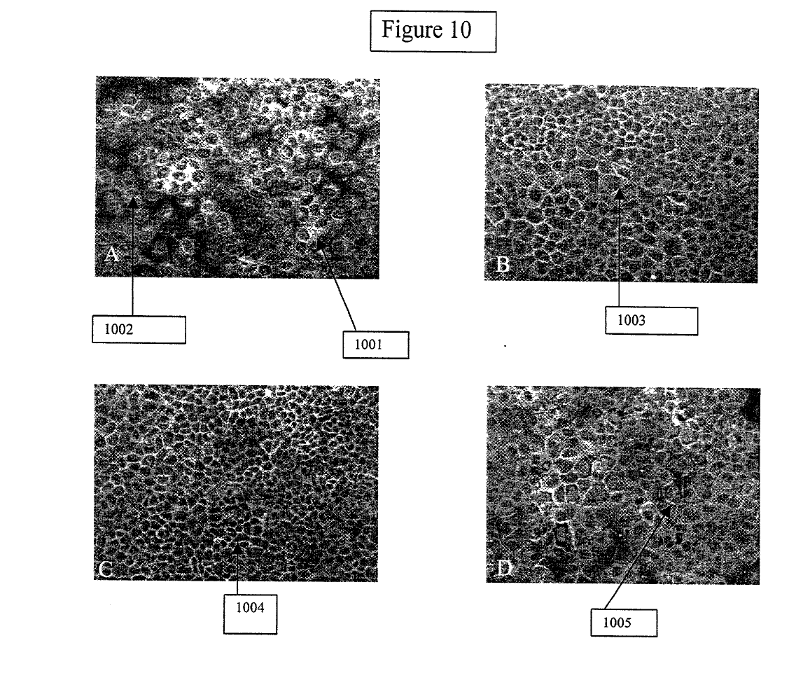

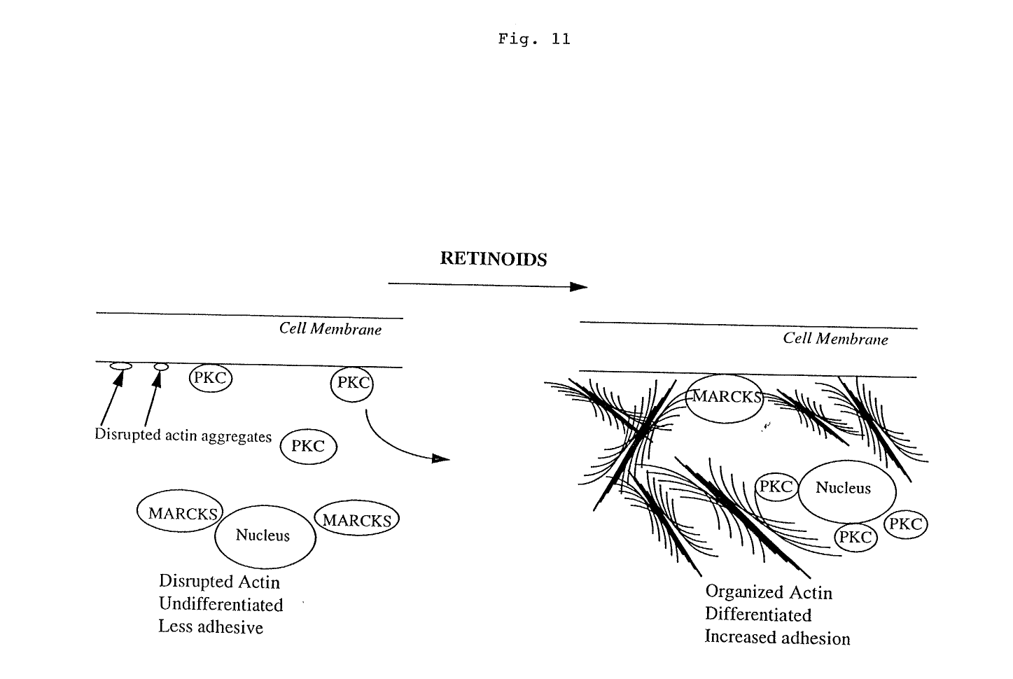

We examined the effects of retinoic acid treatment on MARCKS localization, because MARCKS is a major PKC substrate that crosslinks F-actin when it is localized to the plasma membrane. MARCKS was localized to the cytoplasm with some perinuclear staining in media (FIG. 10A) and vehicle (ethanol) control cells with scattered plasma membrane staining. Upon treatment with 1 μM all-trans (FIG. 10B) or 1 μM 13-cis (FIG. 10C) retinoic acid, MARCKS localized to the plasma membrane. Another set of cells were treated with a PKC inhibitor, bisindolylmaleimide, to down regulate PKC. When cells were treated with bisindolylmaleimide MARCKS staining was prominent in the plasma membrane similar to retinoic acid treated cells (FIG. 10D). However, some scatterred cytoplasmic and perinuclear staining was observed in these cells. Cells that received no primary antibody but only received secondary antibody did not stain. FIG. 11 shows a model of retinoid-induced actin reorganization indicating that the translocation of PKC to perinuclear areas and MARCKS to the plasma membrane allows actin reorganization.

### Discussion

In this study, we have characterized a new cell line of poorly differentiated human endometrial adenocarcinoma cells termed CAC-1 cells and shown that these cells respond to retinoic acid in a similar manner as a previously established cell line, RL95-2 cells (Carter, et al., 1996, Carter and Parham, 1997). We have additionally investigated the effects of retinoid-induced differentiation on PKC-α and MARCKS, a major PKC substrate and have correlated relocalization of these proteins with differentiation evidenced by actin reorganization.

CAC-1 cells are epithelial evidenced by their positive immunostaining for cytokeratin and negative staining for vimentin. It is well known that epithelial cells stain positively for cytokeratins and are negative for vimentin staining (Moll, et al., 1982). CAC-1 cells stained positively for cytokeratin 18. This is not surprising because simple epithelia such as epithelial cells from the endometrium express cytokertain 18. Electron microscopy showed that CAC-1 cells are rounded and that their surface is covered with numerous microvilli. Cells also possessed mitochondria, rough and smooth endoplasmic reticulum and free ribosomes. CAC-1 cells are identifiable from normal endometrial epithelial cells in culture based upon the high nuclear to cytoplasmic ratio, irregularly shaped nuclei and several prominent nucleoli per nucleus. These ultrastructural characteristics are similar to the earlier characterized moderately differentiated human endometrial adenocarcinoma RL95-2 cells (Way, et al., 1983) and to tumors derived from nude mice after the introduction of poorly differentiated KLE cells (Richardson, et al., 1984). CAC-1 cells tend to form colonies of tightly packed cells. Cell function appears to involve protein manufacturing due to the presence of several ribosomes. The presence of desmosomes indicates some mechanical stress and gap junctions indicate communication between cells.

The karyotype of CAC-1 cells showed that there was trisomy of chromosomes 3, 7 and 17. The retinoic acid receptor-α gene is located on chromosome 17. A gain of chromosome 17 has been observed in colorectal cancer and gastric cancer. Increased depth of invasion in colorectal cancers was associated with a higher frequency of a gain of chromosome 17. Likewise, in gastric cancers, the frequency of numerical abberations of chromosome 17 correlated significantly with the depth of invasion and lymph node metastasis. Complex chromosomal abnormalities have been reported in endometrial stromal sarcomas. Four out of 11 endometrial stromal sarcomas exhibited the t(7:17) translocation. Trisomy of chromosome 7 was found in 74% of colorectal tumors. Abberations of chromosome 3 alone or in combination with 7 occurred in HPV-associated cervical cancer. In fact, trisomy of chromosome 7 may be considered an early event in cervical carcinogenesis, persisting and increasing with progression of the lesion. CAC-1 cells have an addition of 7q22. This is of interest because an actin gene has been located on 7q22.

We evaluated the effects of retinoic acid on low passage, poorly differentiated CAC-1 cells. Low passage cells have a greater chance than higher passage cells of exhibiting the same differentiation properties as the tumor from which they were derived in vivo. Further, these CAC-1 cells permitted us to evaluate the retinoid effects on poorly differentiated human endometrial adenocarcinoma derived from the primary tumor. Retinoic acid treatment induced actin filament reorganization and cell enlargement in CAC-1 cells similar to the effect observed earlier in RL95-2 cells (Carter, et al., 1996). Treatment with retinoic acid also induced a slight lag in cell growth in an 8 day growth curve. Because cells with organized actin filaments are often stationary (Pienta, et al., 1989), and adherent, we evaluated the effects of retinoid treatment on cell detachment. Retinoid treatment decreased cell detachment by around 40% compared to media or vehicle controls. This is consistent with other studies which showed that retinoic acid promoted cell adhesion. Retinoic acid (1 μM for 5 days) caused reorganization of F-actin in melanoma cells, promoted cell adhesion, and decreased invasion. Retinoids also enhanced adhesion of F9 teratocarcinoma cells. Retinoic acid treatment inhibited cell growth and increased cell adhesion in MCF-7 cells. Our results are consistent with other studies because retinoic acid treatment of CAC-1 cells caused a decrease in cell growth and reversion to the stationary phenotype evidenced by a dramatic reorganization of actin filaments and cell enlargement, concurrent with a significant decrease in cell detachment.

We have shown that all-trans retinoic acid binds directly to and inactivates PKC-α (Radominska, et al., 2000; J Biological Chemistry). Retinoic acid treatment caused PKC-α to localize exclusively to the cytoplasm in RL95-2 cells, whereas control cells displayed PKC-α localized to the plasma membrane and scatterred diffusely throughout the cytoplasm. The retinoid-induced relocalization of PKC-α to the cytoplasm is consistent with PKC inactivation (Carter, et al., 1998). Because PKC-α is the most common PKC isoform, and is associated with the cytoskeleton, we investigated the effects of retinoic acid treatment on the localization and amount of PKC-α in CAC-1 cells. In control cells, PKC was localized to the plasma membrane and scatterred diffusely throughout the the cytoplasm. Retinoic acid treatment of CAC-1 cells caused PKC-α to localize to cytoplasmic and perinuclear areas. Consistent with this, Western blot analysis showed that PKC-α decreased in the particulate fraction upon retinoid treatment. Because the active form of PKC is located in the membrane (Nishizuka, 1988), a cytoplasmic localization suggests that PKC-α can not translocate to the membrane and become activated upon retinoic acid treatment. Therefore, our results suggest that PKC-α is inactivated by retinoic acid treatment in CAC-1 cells.

MARCKS is an actin-binding protein that cycles between the membrane and cytoplasm. The cycle of membrane attachment and detachment represents a mechanism through which PKC reversibly regulates actin-membrane interactions (Allen and Aderem, 1995). However, this mechanism has not been investigated in human endometrial adenocarcinoma cells. Because MARCKS becomes dephosphorylated and relocalizes to the membrane where it cross-links actin filaments upon PKC inactivation (Allen and Aderem, 1995), we examined the localization of MARCKS in retinoid treated cells. Retinoic acid treatment of CAC-1 cells caused MARCKS staining to increase in the plasma membrane, whereas untreated cells exhibited MARCKS staining in the cytoplasm. When a mutated MARCKS was transfected into fibroblasts, the formation of cell surface extensions and cell spreading was inhibited. This defect in the regulation of the actin cytoskeleton was accompanied by a decrease in cell-substratum adhesion. These results provided direct evidence that MARCKS and PKC regulate actin-dependent membrane ruffling and cell adhesion.

Based on this study and our previous work, we suggest that retinoic acid induces differentiation evidenced by actin filament reorganization and cell enlargement in CAC-1 cells by binding to and inactivating PKC-α causing PKC-α to localize exclusively to the cytoplasm. This inactivation of PKC permits MARCKS to become dephosphorylated and relocalize to the plasma membrane where it can cross-link actin filaments. CAC-1 are in ideal model for the investigation of retinoid induced differentiation of poorly differentiated human endometrial adenocarcinoma cells.

Specimens of CAC-1 cells will be deposited in the American Type Culture Collection or other acceptable depository upon notification that such deposit is essential to the patentability of the invention disclosed herein.

Those skilled in the art who have the benefit of this disclosure will appreciate that it may be used as the creative basis for designing devices or methods similar to those disclosed herein, or to design improvements to the invention disclosed herein; such new or improved creations should be recognized as dependant upon the invention disclosed herein, to the extent of such reliance upon this disclosure.

### List of Citations for Refereneces

1. Aderem, A. (1992). The MARCKS brothers: a family of protein kinase C substrates. Cell 71, 713-716.

2. Allen, L-A H. and Aderem, A. (1995). Protein kinase C regulates MARCKS cycling between the plasma membrane and lysosomes in fibroblasts. EMBO J. 14, 1109-1121.

3. Budd, G. T., Adamson, P. C., Gupta, M., Homayoun, P., Sandstrom, S. K., Murphy, R. F., McLain, D., Tuason, L., Peereboom, D., and Ganapathi, R. D. (1998). Phase I/II trial of all-trans retinoic acid and tamoxifen in patients with advanced breast cancer. Clin. Cancer Res. 4, 635-642.

4. Carter, C. A., Rinehart, C. A., Bagnell, C. R., and Kaufman, D, G. (1991). Fluorescent laser scanning microscopy of F-actin disruption in human endometrial stromal cells expressing the SV40 large T antigen and the EJ ras oncogene. Pathobiology. 59, 36-45.

5. Carter, C. A., Albright, C. D., and Kaufinan, D. G. (1992). Differential effects of dioctanoylglycerol on fibronectin localization in normal, partially transformed, and malignant human endometrial stromal cells. Exp. Cell Res. 201, 262-272.

6. Carter, C. A., Doherty, M. M., Rusnak, D. W., Nettesheim, P., and Ferriola, P. C. (1994). Alterations in the localization of F-actin, fibronectin and thrombospondin occur early in multistep transformation of rat tracheal epithelial cells. Exp. Cell Res. 212, 141-150.

7. Carter, C. A., Pogribny, M., Davidson, A., Jackson, C. D., McGarrity, L. J., and Morris, S. M. (1996). Effects of retinoic acid on cell differentiation and reversion toward normal in human endometrial adenocarcinoma (RL95-2) cells. Anticancer Res. 16, 17-24.

8. Carter, C. A., and Parham, G. P. (1997). State of differentiation affects the response of endometrial adenocarcinoma cells to retinoic acid. Anticancer Res. 17,1973-1984.

9. Carter, C. A., Parham, G. P., and Chambers, T. C. (1998). Cytoskeletal reorganization induced by retinoic acid treatment of human endometrial adenocarcinoma (RL95-2) cells is correlated with alterations in protein kinase C-α. Pathobiology. 66, 284-292.

10. Carter, C. A. and Madden, V. (2000). A newly characterized human endometrial. adenocarcinoma cell line (CAC-1) differentiates in response to retinoic acid treatment. Expt. and Molecular Pathology, 69, 175-191.

11. Fujimoto, J., Hori, M., Ichigo, S., Morishita, S. and Tamaya, T. (1995). Clinical implications of fos and jun expressions and protein kinase activity in endometrial cancers. Eur. J. Gynaecol. Oncol. 16: 138-146.

12. Guo, W. X., Gill, P. S. and Antakly, T. (1995). Inhibition of AIDS-Kaposi's sarcoma cell proliferation following retinoic acid receptor activation. Cancer Res. 55, 823-829.

13. Holinka, C. F., Hata, H., Kuramoto, H., and Gurpide, E. (1986). Effects of steroid hormones and antisteroids on alkaline phosphatase activity in human endometrial cancer cells (Ishikawa line). Cancer Res. 46, 2771-2774.

14. Kudelka A P, Freedman R S, Edwards C L, Lippman S M, Tornos C S, Krakoff I H and Kavanagh J J. (1993). Metastatic adenocarcinoma of the endometrium treated with 13-cis-retinoic acid plus interferon-α. Anti-Cancer Drugs 4: 335-337.

15. Nishizuka, Y. (1988). The molecular heterogeneity of protein kinase C and its implications for cellular regulation. Nature. 334, 661-665.

16. Nishizuka, Y. (1989). The family of protein kinase C for signal transduction. JAMA 262, 826-1833.

17. Pienta, K. J., Partin, A. W., and Coffey, D. S. (1989). Cancer as a disease of DNA organization and dynamic cell structure. Cancer Res. 49, 2525-2532.

18. Radominska-Pandya, A., Chen, G., Czernik, P. J., Little, J. M., Samokyszyn, V. M., Carter, C. A. and Nowak, G. (2000). Direct interaction of all-trans-retinoic acid with protein kinase C: Implications for PKC signaling and cancer therapy. J. Biological Chemistry. 275, 22324-22330.

19. Rao, J. Y., Hurst, R. E., Bales, W. D., Jones, P. L., Bass, R. A., Archer, L. T., Bell, P. B., and Hemstreet, G. P. (1990). Cellular F-actin levels as a marker for cellular transformation: Relationship to cell division and differentiation. Cancer Res. 50, 2215-2220.

20. Thelen, M., Rosen, A., Naim, A. C. and Aderem, A. (1991). Regulation by phosphorylation of reversible association of a myristoylated protein kinase C substrate with the plasma membrane. Nature 351, 320-322.

21. Richardson G. S., Dickersin, G. R., Atkins, L., MacLaughlin, D. T., Raam, S., Merk, L. P., and Bradley, F. M. (1984). KLE: A cell line with defective estrogen receptor derived from undifferentiated endometrial cancer. Gynecol Oncol 17, 213-230.

22. Tornus, C., and Elvio, S. (1993). Pathology and grading of endometrial adenocarcinomas. Clinical Consultations in Obstet. and Gynecol. 5, 87-95.

23. Way, D. L., Grosso, D. S., Davis, J. R., Surwit, E. A., and Christian, C. D. (1983). Characterization of a new human endometrial carcinoma (RL95-2) established in tissue culture. In Vitro. 19, 147-158.

I claim:

1. A cellular composition, comprising cells isolated from a poorly differentiated uterine cancer.

2. A cellular composition as described in claim 1 hereinabove, wherein said cancer comprises endometrial cancer.

3. A cellular composition as described in claim 1 hereinabove, wherein said cancer comprises adenocarcinoma.

4. A cellular composition as described in claim 1 hereinabove, said cancer having characteristics consistent with a primary tumor.

5. A cellular composition as described in claim 1 hereinabove, wherein said cancer is metastatic.

6. A cell line as described in claim 1 hereinabove, wherein a plurality of said cells have at least 48 chromosomes.

7. A cell line as described in claim 1 hereinabove, wherein a plurality of said cells are at least triploid at chromosomes 3, 7 and 17, but only haploid at chromosome 14.

8. A cellular composition described in claim 1 hereinabove, wherein said cells are grown in vitro as a monolayer.

9. A human endometrial adenocarcinoma cell line described in claim 1 hereinabove, wherein a plurality of said cells have at least the following karyotypic characteristics: 48, XX, ?t (1:20) (p?34.3; p11.2), dup (2) (q11.1q23), +3, del (5) (q?23q?31), ?add(6) (p23), add (7) (p?21), +add (7) (q22), der(9;14) (q10;q10), add (15) (p11), +der (17) t(17,;19) (p11.1;p11.1), I (19) (q10), ?del (20) (p?11.2).

10. A line of cells originating from a specimen of human endometrial adenocarcinoma wherein a plurality of said cells behaves in substantially equivalent ways at the morphological, physiological or molecular levels as a cell of said sample.

11. A method of culturing cells in vitro, comprising the steps introducing said cells to media comprising a 1:1 mixture of Medium 199 and Ham's F12 supplemented with up to 4% serum, antibiotics, anti-micotics and growth factors; and culturing said cells under conditions for proliferation.

12. A method of culturing cells described in claim 11 hereinabove, comprising the steps of:
a. suspending cells, separated from a specimen cancer tissue, in a digestion media including (comprising) a mixture of collagenase A in a growth media comprising Medium 199 and Ham's F12 supplemented with fetal bovine serum, bovine calf serum, penicillin streptomycin, L-glutamine, fungizone and insulin transferrin selenium,
b. centrifuging said suspension until pellet formation,
c. introducing said pellet to a growth media including media comprising Medium 199 and Ham's F12 supplemented with fetal bovine serum, bovine calf serum, penicillin streptomycin, L-glutamine, fungizone and insulin transferrin selenium,
d. centrifuging said introductory solution, and
e. plating a supernatant onto a plating dish including culture media comprising Medium 199 and Ham's F12 supplemented with fetal bovine serum, bovine calf serum, penicillin streptomycin, L-glutamine, fungizone and insulin transferrin selenium.

13. A method of culturing a cell line described in claim 1 hereinabove, comprising the steps of:
a. obtaining a hysterectomy specimen of endometrial adenocarcinoma, and placing said specimen in media 1 including Leibovitz L-15 media, 5% penicillin/streptomycin, 500 μg/ml gentamicin and 2.5 μg/ml fungizone;
b. washing said specimen with Hank's Balanced Salt Solution (“HBSS”);
c. mincing said specimen into 1 mm pieces, with sterile blades;
d. washing said pieces with HBSS;
e. centrifuging said pieces for 3 minutes at 700 rpm, until pellet formation;
f. exposing said pellet to 2 μg/ml of collagenase A in growth media 2 comprising a 1:1 mixture of Medium 199 and Ham's F12 supplemented with 1% fetal bovine serun, 3% bovine calf serum, 5% penicillin streptomycin, 4 mM L-glutamine, 2.5 μg/ml fungizone and 0.1% insulin transferrin selenium, for 1 hr at 37° C., pipetting vigorously every 15 minutes;
g. centrifuging at 700 rpm for 3 minutes, resulting formation of a pellet and a first supernatant;
h. plating said first supernatant onto at least one 60 mm plating dish including growth media 2;
i. re-exposing said pellet to said collegenase A in said growth media 2, for 1 hr at 37° C., pipetting vigorously every 15 minutes, resulting in formation of a second supernatant;
j. plating said second supernatant onto at least one 60 mm plating dish including growth media 2, for growth of a plurality of cells;
k. following approximately one month in said culture, and using a sterile pipette under sterile conditions, gently scraping a plurality of cells from said dish and re-plating said cells onto at least one 60 mm plating dish including growth media 2, for growth of a plurality of cells;
l. after approximately 1 week in said culture, transferring a plurality of cells to at least one 100 mm plating dish including growth media 2, and maintaining said cells in said media until cells become essentially confluent therein; and
m. approximately every 3 to 4 days, splitting the cells of said dish into fractions, then transferring each fraction of same into one of a plurality (approximately 4) of plating dishes including growth media 2, and maintaining said cells in said media until formation of additional cells, repeating as many times as desired.

14. A method of identifying a compound that inhibits the activity of a protein kinase in a cell, comprising the steps of:
a. providing a cell of claim 1 hereinabove,
b. contacting said cell with at least one inhibitor test compound, and
c. determining whether a protein kinase primarily localizes away from the cell membrane, said localization being an indication that said test compound likely inhibits said protein kinase.

15. A method described in claim 14 hereinabove, wherein:
a. said protein kinase is an isoform known to be involved in hindering the organization of cytoskeleton matrix in the cell cytoplasm, and
b. determining whether said isoform localizes primarily away from the cell membrane, said localization being an indication that said cell is apt to undergo organization of cytoskeleton matrix in the cell cytoplasm.

16. A method described in claim 15 hereinabove, wherein:
a. said protein kinase is PKC-α and said inhibitor test compound is retinoic acid, and
b. determining whether PKC-α localizes primarily in a cytoplasmic and perinuclear region, said localization being an indication that said cell is apt to undergo organization of cytoskeleton matrix in the cell cytoplasm.

17. A method of determining the effect of a protein kinase inhibitor on a condition in a cell having manifestations consistent with cancer, comprising the steps of:
a. providing a cell of claim 1 hereinabove,
b. contacting said cell with at least one inhibitor of protein kinase known to be present in abnormally high levels in cells failing to undergo organization of cytoskeleton matrix in the cell cytoplasm, and
c. determining whether protein kinase primarily localizes away from the cell membrane, said localization being an indication that said cell is apt to undergo organization of cytoskeleton matrix in the cell cytoplasm.

18. A method described in claim 17 hereinabove, wherein:
a. said protein kinase is PKC-α and said inhibitor of protein kinase is retinoic acid, and
b. determining whether PKC-α localizes primarily in a cytoplasmic and perinuclear region, said localization being an indication that said cell is apt to undergo organization of cytoskeleton matrix in the cell cytoplasm.

19. A method, using a cell isolated in vitro, for predicting the effect on cell differentiation attributable to a differentiation enhancing test compound to be applied to an in vivo cancer cell, comprising the steps of:
a. providing a cell of claim 1 hereinabove,
b. contacting said cell with at least one enhancer test compound, and
c. determining whether actin filaments organize a cytoskeleton matrix, said organization being an indication that said test compound likely enhances cell differentiation.

20. A method as described in claim 19 hereinabove, wherein said enhancer test compound is retinoic acid.
